(19) United States
(12) Patent Application Publication
(10) Pub. No.: US 2008/0199414 A1
(43) Pub. Date: Aug. 21, 2008

(54) COSMETIC FORMULATION COMPRISING FLAVONOID DERIVATIVES

(76) Inventors: Corinna Wirth, Darmstadt (DE); Herwig Buchholz, Frankfurt (DE); Christophe Carola, Langen (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/107,526

(22) Filed: Apr. 22, 2008

Related U.S. Application Data
(63) Continuation of application No. 10/468,708, filed on Aug. 21, 2003.

(30) Foreign Application Priority Data
Mar. 2, 2001 (DE) 101 10 105.8

(51) Int. Cl. A61K 8/60; C07H 15/26; A61Q 17/04; A61P 29/00; A61K 31/7048

(52) U.S. Cl. 424/59; 536/18.1; 514/27

(57) ABSTRACT

The invention relates to cosmetic formulations, pharmaceutical preparations, food products and food supplements containing flavonoid derivatives. The flavonoid derivatives act therein, for instance, as UV filter. Some flavonoid derivatives represent novel compounds.

The invention relates to cosmetic formulations comprising flavonoid derivatives, to the use of these compounds, in particular in cosmetic formulations, and to novel UV-active compounds.

A certain degree of tanning of the skin is regarded in modern society as attractive and as an expression of vigour and sportiness. In addition to this desired action of the sun on the skin, a number of undesired side effects occur, such as sunburn or premature skin ageing and wrinkling. A number of effective UV filters have now been developed which, applied to the skin in the form of creams, lotions or gels, are able effectively to delay the development of sunburn, even in the case of relatively great exposure to the sun. The UV filter present in the pharmaceutical or cosmetic preparation forms a film or layer on the surface of the skin and does not penetrate into deeper skin layers with further care substances present in the preparation. Known UV filters and sunscreens thus act by absorbing certain regions of the sunlight, thus preventing this radiation from penetrating into deeper layers of the skin. As is known, the most dangerous part of solar radiation is formed by ultraviolet rays having a wavelength of less than 400 nm. The lower limit for the ultraviolet rays which reach the earth's surface is restricted to about 280 m by absorption in the ozone layer. The sun-protection filters usual today in cosmetics absorb in a wavelength range from 280 to 400 m. This range covers UV-B rays having a wavelength of between 280 and 320 m, which play a crucial role in the formation of solar erythema, and also UV-A rays having a wavelength of between 320 and 400 m, which tan the skin, but also allow ageing, favour the triggering of an erythematous reaction or can exacerbate this reaction in certain people or even trigger phototoxic or photoallergic and irritative reactions.

Skin damage is not caused just by sunlight, but also by other external influences, such as cold or heat. Furthermore, the skin undergoes natural ageing, with the formation of wrinkles and a reduction in the elasticity of the skin.

The object of care cosmetics is wherever possible to obtain the impression of youthful skin. In principle, there are various ways of achieving this aim. For example, existing skin damage, such as irregular pigmentation or the development of wrinkles, can be compensated for by covering powders or creams. Another approach is to protect the skin against environmental influences which lead to permanent damage and thus ageing of the skin. The idea is therefore to intervene in a preventative manner and thus to delay the ageing process. One example of this is the UV filters already mentioned, which, as a result of absorption of certain wavelength ranges, prevent or at least reduce skin damage. Corresponding to the position of their absorption maxima, UV absorbers for cosmetic and dermatological preparations are divided into UV-A and UV-B absorbers, with UV-A absorbers usually also absorbing in the UV-B region and therefore alternatively also being referred to as broad-band absorbers or filters.

However, the known UV filters often have disadvantages: for example, they are not tolerated by the skin to a satisfactory extent or their absorption properties are inadequate. The inadequate absorption properties may be evident, for example, from the fact that only a small part of the UV spectrum is absorbed or that the absorption coefficient at a given wavelength is unsatisfactory.

While in the case of UV filters the harmful event, the UV radiation, is screened away from the skin, it is attempted in another method to support the natural defence and repair mechanisms of the skin against the harmful event. Finally, as a further approach, it is attempted to compensate for the weakening defence functions of the skin against harmful influences with increasing age by the external supply of substances which are able to replace this diminishing defence or repair function. For example, the skin has the ability to scavenge free radicals generated by external or internal stress factors. This ability weakens with increasing age, causing the ageing process to accelerate with increasing age.

A further difficulty in the preparation of cosmetics is that active ingredients which are intended to be incorporated into cosmetic formulations are frequently unstable and can be damaged in the formulation. The damage may be caused, for example, by a reaction with atmospheric oxygen or by absorption of UV rays. The molecules damaged in this way may, for example, change their colour and/or lose their activity through their structural change.

The object of the present invention was therefore to provide cosmetic formulations which avoid the disadvantages of the prior art and have, in particular, advantageous absorption properties.

Surprisingly, it has now been found that this object is achieved by the use of the compounds of the formula I

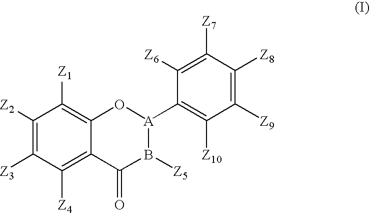

in which
Z₁ to Z₄ and
Z₆ to Z₁₀ are each, independently of one another, H, OH, CH₃COO, alkoxy, hydroxyalkoxy, mono- or oligoglycoside radicals and where the alkoxy and hydroxyalkoxy groups may be branched or unbranched and can have from 1 to 18 carbon atoms,

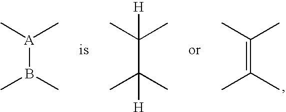

Z₅ is a mono- or oligoglycoside radical, where at least one radical selected from

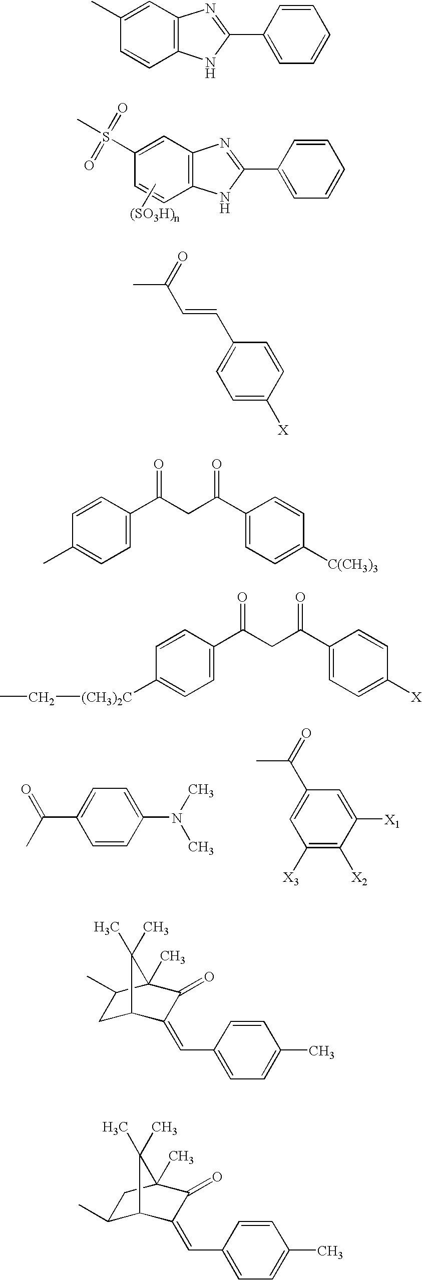

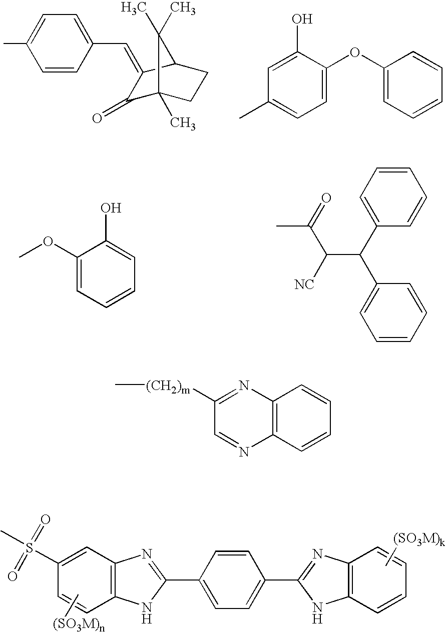

in which X, X₁, X₂ and X₃ are each, independently of one another, OH, CH₃COO, an alkoxy radical having from 1 to 8 carbon atoms or a monoglycoside radical, n is 0, 1, 2 or 3, m is 0 or 1, k is 0, 1, 2, 3 or 4, and M is H, Na or K,
is bonded to this glycoside radical, in each case via an —O-group, and
   in which one or more hydrogen atoms in the OH groups of the glycoside radicals mentioned in the substituents Z₁ to Z₁₀ may each, independently of one another, also be replaced by acetyl or by alkyl radicals having from 1 to 8 carbon atoms, and where, in each case independently of one another, sulfate or phosphate may also be bonded to one or more hydroxyl groups of the radicals mentioned in the substituents Z₁ to Z₁₀,

in cosmetic formulations.

The invention thus relates to cosmetic formulations comprising one or more compounds of the formula I

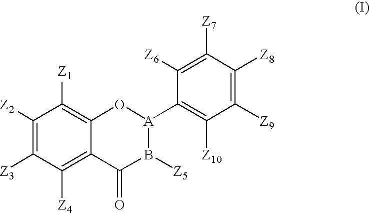

in which
Z₁ to Z₄ and
Z₆ to Z₁₀ are each, independently of one another, H, OH, CH₃COO, alkoxy, hydroxyalkoxy, mono- or oligoglycoside radicals and where the alkoxy and hydroxyalkoxy groups may be branched or unbranched and can have from 1 to 18 carbon atoms,

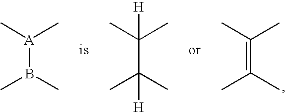

Z₅ is a mono- or oligoglycoside radical, where at least one radical selected from

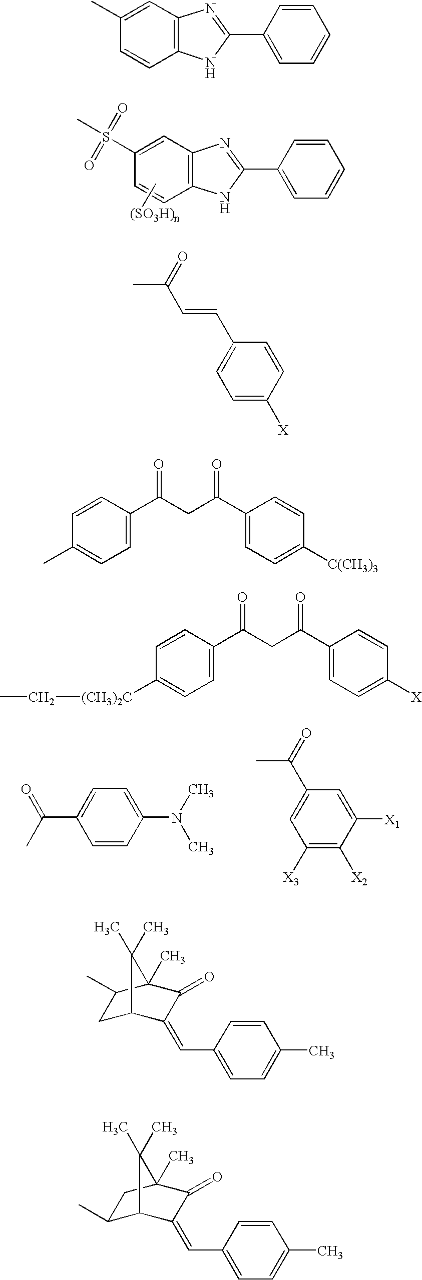

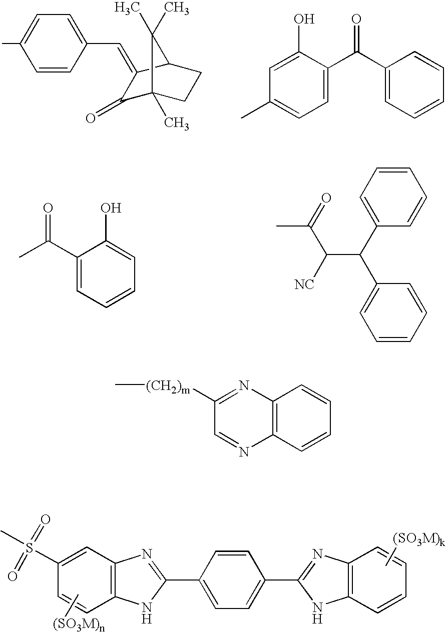

in which X, X₁, X₂ and X₃ are each, independently of one another, OH, CH₃COO, an alkoxy radical having from 1 to 8 carbon atoms or a monoglycoside radical, n is 0, 1, 2 or 3, m is 0 or 1, k is 0, 1, 2, 3 or 4, and M is H, Na or K,
is bonded to this glycoside radical, in each case via an —O-group, and
   in which one or more hydrogen atoms in the OH groups of the glycoside radicals mentioned in the substituents Z₁ to Z₁₀ may each, independently of one another, also be replaced by acetyl or by alkyl radicals having from 1 to 8 carbon atoms, and where, in each case independently of one another, sulfate or phosphate may also be bonded to one or more hydroxyl groups of the radicals mentioned in the substituents Z₁ to Z₁₀.

Compounds of the formula I have already been disclosed. However, they have to date not been used in cosmetic formulations.

Known compounds of the formula I are, for example, kaempferol 3-(6″-galloylglucoside) and kaempferol 3-(6″-p-coumarylglucoside), which is also known as tiliroside.

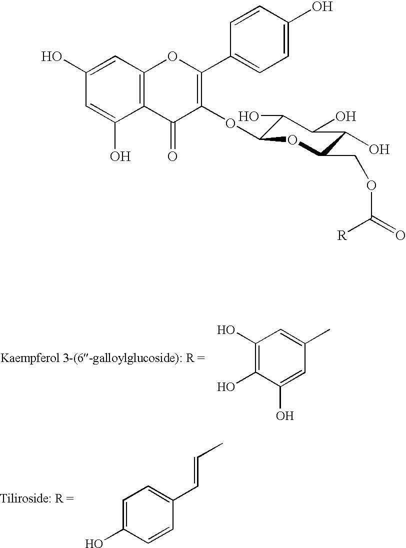

DE 195 44 905 A1 describes, for example, a process for the preparation of plant extracts containing tiliroside and the use of the plant extracts in medicaments and food products. However, cosmetic formulations comprising tiliroside are not described in DE 195 44 905 A1.

DE 199 22 287 A1 describes tiliroside as a starting flavonoid for the preparation of tiliroside esters whose acid unit contains from 3 to 30 carbon atoms. These esters are used in cosmetics. However, DE 199 22 287 A1 does not describe any cosmetic formulations comprising tiliroside.

The cosmetic formulations according to the invention comprising one or more compounds of the formula I have, for example, the advantage that they absorb both in the UV-A and in the UV-B region. This means that broad-band UV protection can be achieved through the use of the formulations according to the invention. In addition, the formulations according to the invention have good absorption coefficients. This is illustrated below using the example of the substance tiliroside.

Tiliroside absorbs in the UV-B region, i.e. in the wavelength range between 290 and 320 nm, with an absorption maximum at λₘₐₓ=316 nm, and additionally in the UV-A region, i.e. in the wavelength range between 320 and 400 nm, with an absorption shoulder at λₛₕ=349 nm. The absorption coefficient ε at λₘₐₓ=316 nm is ε=23260 and at λₛₕ=349 nm is ε=13500.

The advantageous UV-absorbing properties of the compounds of the formula I present in the cosmetic formulations according to the invention become particularly clear if they are compared with those of other substances which are commercially available and are used in sunscreen formulations. For example, Eusolex® 6300

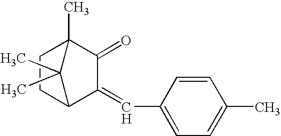

Eusolex 6300 exhibits an absorption maximum at λₘₐₓ=300 nm in the UV-B region. The absorption coefficient at this wavelength is 23420. However, Eusolex® 6300 does not absorb in the UV-A region. This means that tiliroside and Eusolex® 6300 have a comparable absorption coefficient in the UV-B region, while tiliroside has significantly better absorption properties in the UVA region.

The present invention therefore also relates to the use of one or more compounds of the formula I as UV filters, in particular in cosmetic formulations.

Besides the advantageous UV-absorbing properties, the compounds of the formula I additionally exhibit advantageous antioxidant and free-radical-scavenging properties. The present invention thus furthermore also relates to the use of one or more compounds of the formula I as free-radical scavengers and/or antioxidants, in particular in cosmetic formulations.

Through their action as antioxidant, compounds of the formula I also have a stabilising action on formulations used, for example, in cosmetics. Through the addition of compounds of the formula I to the corresponding products, the latter therefore remain stable for longer and do not change their appearance. In particular, the effectiveness of the ingredients is also retained on extended application or extended storage. This is particularly advantageous in the case of sunscreens, since these cosmetics are subjected to particularly high exposure to UV rays.

The formulations comprising one or more compounds of the formula I are particularly suitable for the protection of human skin or for the protection of body cells against oxidative stress, i.e., for example, against damage by free radicals, as generated, for example, by sunlight, heat or other influences. The formulations comprising one or more compounds of the formula I are particularly suitable for reducing skin ageing.

The present invention thus also relates to the use of one or more compounds of the formula I as active ingredient for protection against oxidative stress, in particular in cosmetic formulations. The present invention furthermore relates to the use-of one or more compounds of the formula I for preventing skin ageing, in particular in cosmetic formulations.

The compounds of the formula I additionally have antiallergic, antiinflammatory, inflammation-inhibiting and antiirritative properties and can thus be used for the treatment or preventive treatment of allergies, inflammation and irritation, in particular of the skin. The present invention therefore furthermore relates to the use of one or more compounds of the formula I as active ingredient having an antiallergic, antiinflammatory, inflammation-inhibiting and antiirritative action; in particular in cosmetic formulations.

Furthermore, compounds of the formula I, such as, for example, tiliroside, have only a weak inherent colour. The weak inherent colour is, for example, a major advantage if an inherent colour of the ingredients is undesired in the products for aesthetic reasons.

In the compounds of the formula I, the alkoxy, groups are preferably linear and have from 1 to 12 and preferably from 1 to 8 carbon atoms. These groups thus conform to the formulae —O—(CH₂)ₘ—H, where m is 1, 2, 3, 4, 5, 6, 7 or 8 and in particular from1 to 5.

In the compounds of the formula I, the hydroxyalkoxy groups are preferably linear and have from 2 to 12 and preferably from 2 to 8 carbon atoms. These groups thus conform to the formulae —O—(CH₂)ₙ—OH, where n is 2, 3, 4, 5, 6, 7 or 8, in particular from 2 to 5 and extremely preferably 2.

If one or more of the radicals Z₁ to Z₄ and Z₆ to Z₁₀ in the compounds of the formula I are a mono- or oligoglycoside radical, this glycoside radical is bonded directly to the corresponding benzene ring in the formula I via an oxygen atom. The mono- or oligoglycoside radicals are preferably built up from 1 to 3 glycoside units. These units are preferably selected from the group consisting of hexosyl radicals, in particular rhamnosyl radicals and glucosyl radicals. However, other hexosyl radicals, for example allosyl, altrosyl, galactosyl, gulosyl, idosyl, mannosyl and talosyl, may also advantageously be used. It may also be advantageous in accordance with the invention to use pentosyl radicals.

The mono- or oligoglycoside radicals present in the radical Z₅ of the compounds of the formula I are bonded to the group “B” of the formula I via an oxygen atom and are preferably built up from 1 to 3 glycoside units. The preferred units in the radicals Z₁ to Z₄ and Z₆ to Z₁₀ are also preferred for the mono- or oligoglycoside radical present in the radical Z₅. The mono- or oligoglycoside radical present in the radical Z₅ is particularly preferably selected from the group consisting of the radicals of glucose, rhamnose and rutinose.

If X, X₁, X₂ and/or X₃ in the compounds of the formula I are a monoglycoside radical, these glycoside radicals are each bonded to the corresponding benzene ring via an oxygen atom. The preferred units in the radicals Z₁ to 14 and, Z₆ to Z₁₀ are also preferred for this monoglycoside radical. If X, X₁, X₂ and/or X₃ are a monoglycoside radical, the glucose radical is particularly preferred.

In a preferred embodiment of the invention, in particular if the water solubility of the compounds of the formula I is to be increased, a polar group, for example, in each case independently of one another, a sulfate or phosphate group, is bonded to one or more hydroxyl groups of the radicals mentioned in the substituents Z₁ to Z₁₀. Suitable counterions are, for example, the ions of the alkali or alkaline earth metals, these being selected, for example, from sodium and potassium.

In a further preferred embodiment of the invention, preference is given to the compounds of the formula I in which the radicals having an aromatic component which are present in the substituent Z₅ are bonded to the mono- or oligoglycoside radical likewise present in the radical Z₅ via an ester group —OOC—.

In a further preferred embodiment of the invention, sub-formulae of the formula I are derived from the compounds from the following group: rutin, trishydroxyethylrutin (troxerutin), isoquercetin, trishydroxyethylisoquercetin (troxeisoquercetin) and astragalin, and the sulfates and phosphates thereof.

In a further preferred embodiment, the compounds of the formula I present in the formulations according to the invention are selected from the compounds of the formula IA

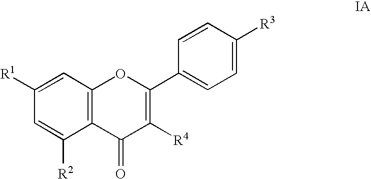

in which
R¹, R² and R³ are each, independently of one another, OH, CH₃COO, an alkoxy radical having from 1 to 8 carbon atoms or a monoglycoside radical,
R⁴ is a mono- or diglycoside radical, where at least one group selected from

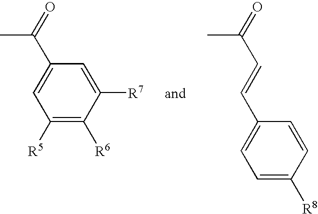

is bonded to the glycoside radical, in each case via an —O-group,
R⁵, R⁶, R⁷
and R⁸ each, independently of one another, have the meaning of the radicals R¹ to R³, and
   in which one or more hydrogen atoms in the OH groups of the glycoside radical(s) may each, independently of one another, also be replaced by acetyl or by alkyl radicals having from 1 to 8 carbon atoms, and where, in each case independently of one another, sulfate or phosphate may also be bonded to one or more hydroxyl groups of the compounds of the formula IA.

In a preferred embodiment, the radical R² in the compounds of the formula IA is selected from OH, CH₃COO and an alkoxy radical having from 1 to 8 carbon atoms.

In the compounds of the formula IA, all OH groups of the mono- or diglycoside radical of R⁴ may be esterified with a group of the formula

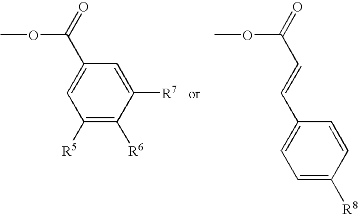

Preferably, however, only one or two of the radicals derived from these radicals are bonded to the glycoside radical.

If R⁴ is a mono- or diglycoside radical in which one or more hydrogen atoms of the OH groups have been replaced by acetyl or alkyl radicals, all OH groups for which replacement is possible have then preferably been replaced by acetyl or alkyl.

Of the alkoxy radicals having from 1 to 8 carbon atoms mentioned in the compounds of the formula IA, the methoxy group is preferred. Of the alkyl radicals having from 1 to 8 carbon atoms mentioned in the compounds of the formula IA, the methyl group is preferred.

The mono- and diglycoside radicals mentioned in the compounds of the formula IA are preferably built up from glucose units.

Preferred compounds IA1 to IA13 selected from the compounds of the formula IA are indicated below:

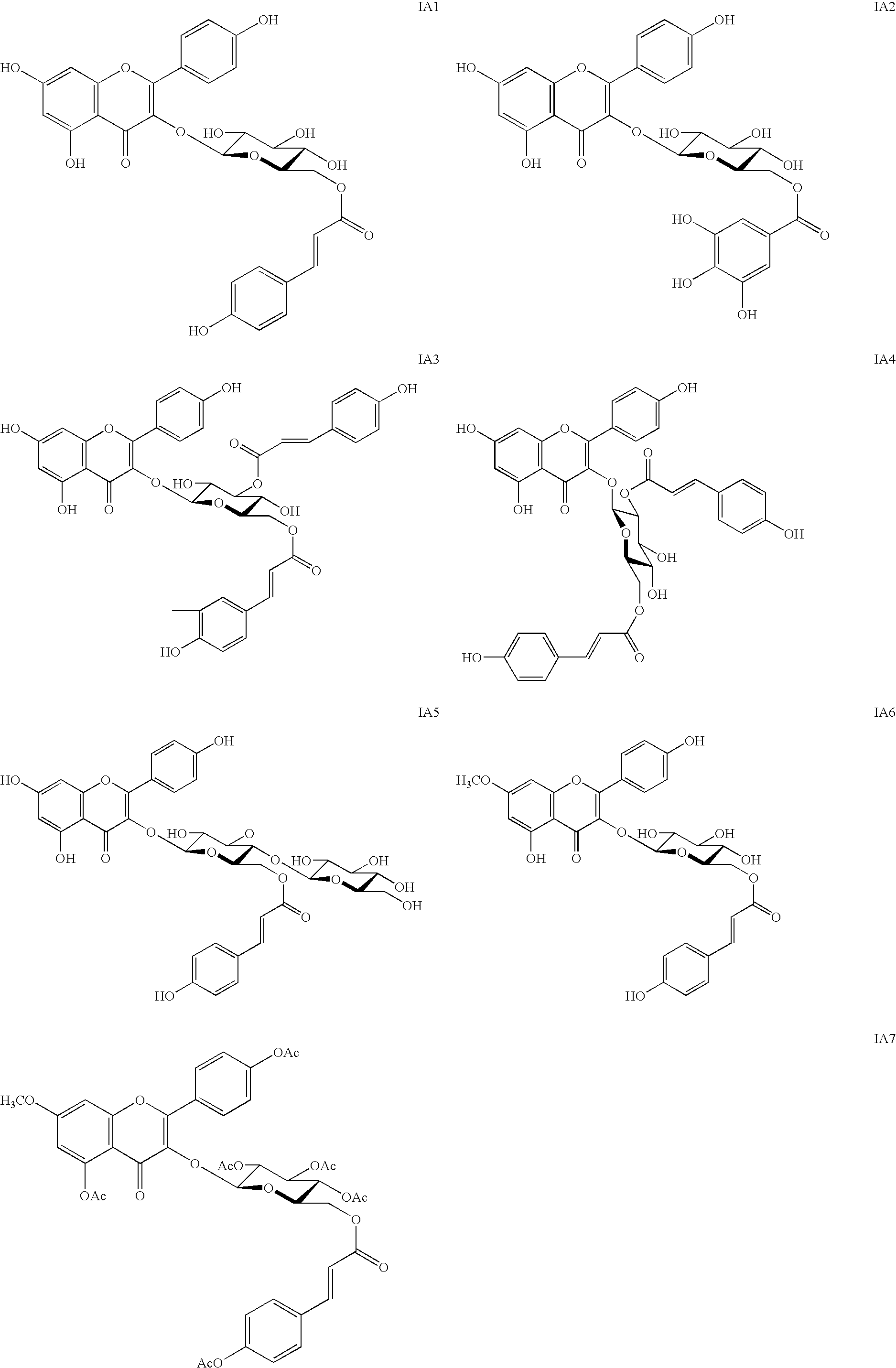

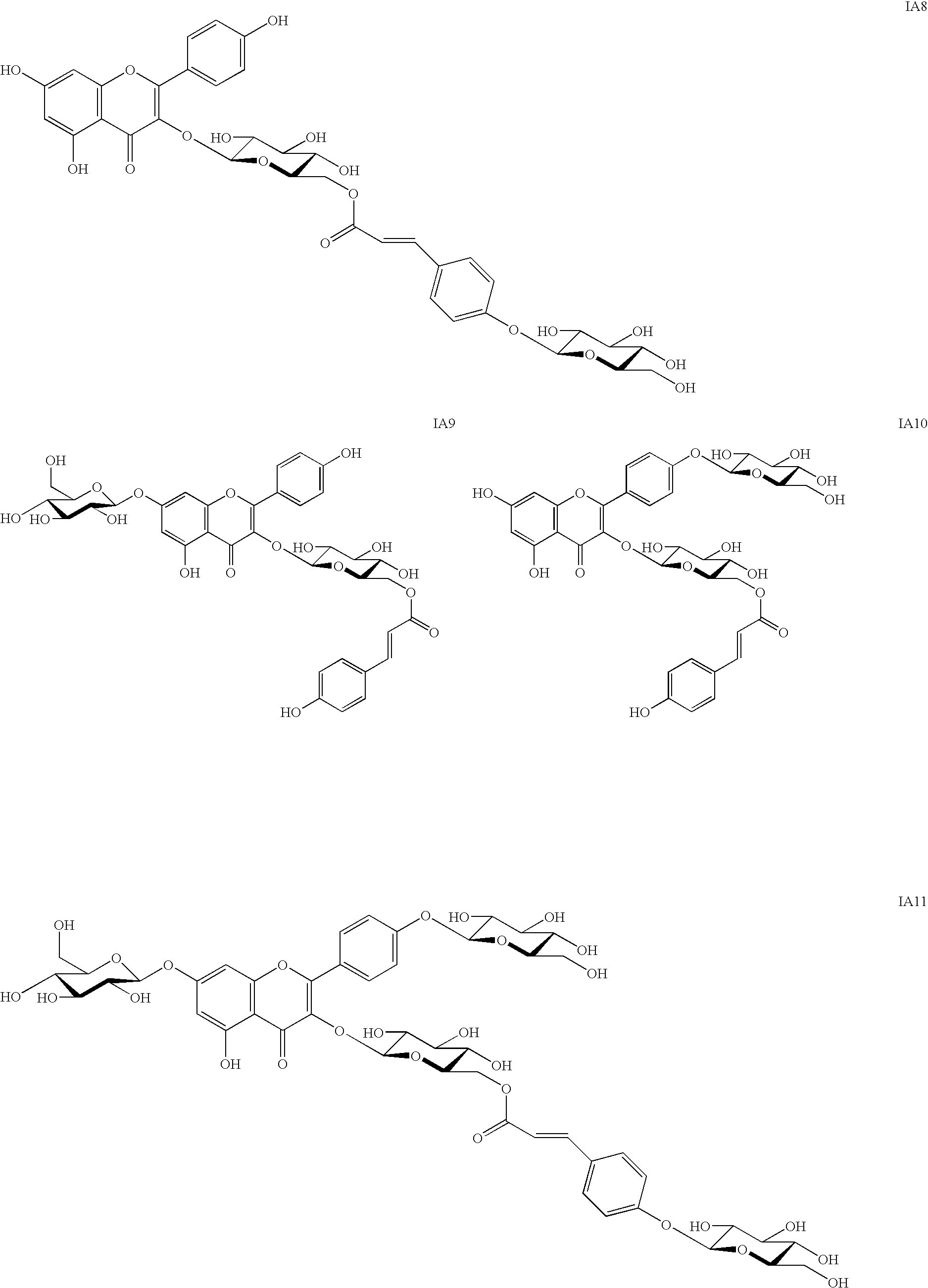

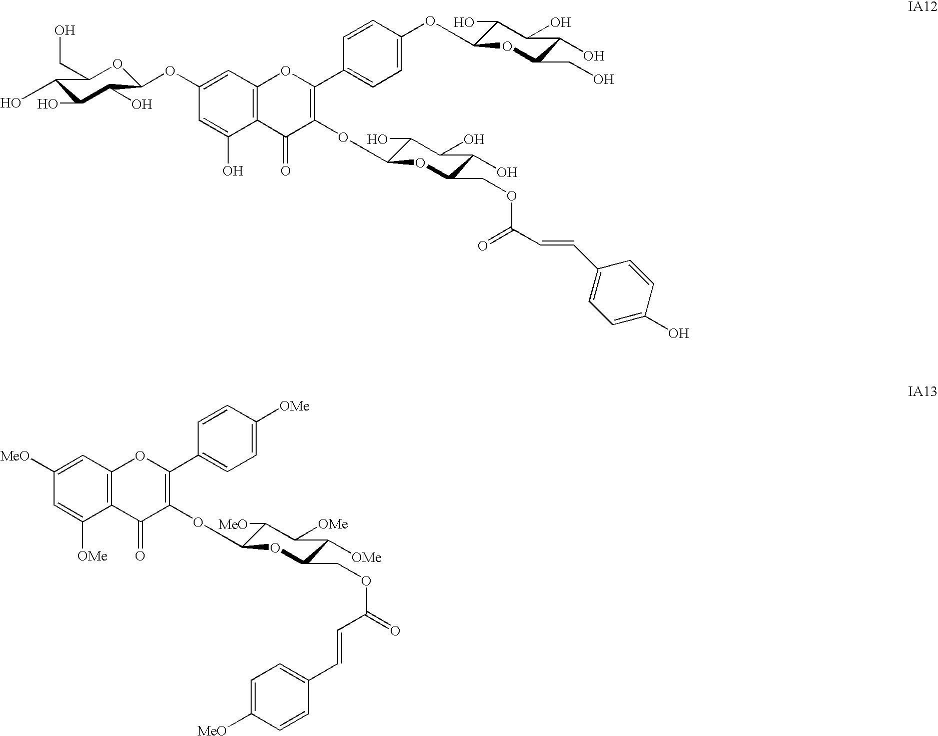

in the compounds of the formulae IA1 to IA13 mentioned above, Me is methyl and Ac is acetyl.

Of the compounds of the formula IA, particular preference is given to the compounds of the formulae IA1 and IA2. Very especial preference is given to the compound of the formula IA1, i.e. tiliroside.

In a further preferred embodiment, the compounds of the formula I present in the formulations according to the invention are selected from the compounds in which

Z₁ to Z₄ and Z₆ to Z₁₀ are each, independently of one another, H, OH, alkoxy, hydroxyalkoxy, mono- or oligoglycoside radicals and where the alkoxy and hydroxyalkoxy groups may be branched or unbranched and can have from 1 to 18 carbon atoms,

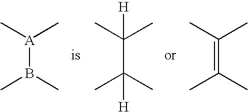

Z₅, n, m, k and M are as defined in claim 1, but the radicals X, X₁, X₂ and X₃ present in the substituent Z₅ are each, independently of one another, OH, an alkoxy radical having from 1 to 8 carbon atoms or a monoglycoside radical,

and in which one or more hydrogen atoms in the OH groups of the glycoside radicals mentioned in the substituents Z₁ to Z₁₀ may each, independently of one another, also be replaced by alkyl radicals having from 1 to 8 carbon atoms, and where, in each case independently of one another, sulfate or phosphate may also be bonded to one or more hydroxyl groups of the radicals mentioned in the substituents Z₁ to Z₁₀.

In these compounds of the formula I, Z₁ to Z₄ and Z₈ to Z₁₀ are preferably each, independently of one another, H, OH, alkoxy or hydroxyalkoxy.

In a further preferred embodiment, the compounds of the formula IA present in the formulations according to the invention are selected from the compounds in which
R¹, R² and R³ are each, independently of one another, OH, an alkoxy radical having from 1 to 8 carbon atoms or a monoglycoside radical,
R⁴ is a mono- or diglycoside radical, where at least one group selected from

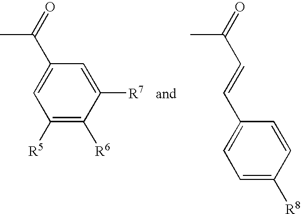

is bonded to the glycoside radical, in each case via an —O-group,
R⁵, R⁶, R⁷
and R⁸ are each, independently of one another, OH, an alkoxy radical having from 1 to 8 carbon atoms or a monoglycoside radical, and
   in which one or more hydrogen atoms in the OH groups of the glycoside radical(s) may each, independently of one another, also be replaced by alkyl radicals having from 1 to 8 carbon atoms, and where, in each case independently of one another, sulfate or phosphate may also be bonded to one or more hydroxyl groups of the compounds of the formula IA.

In these compounds of the formula IA, R¹ to R³ are preferably each, independently of one another, OH or an alkoxy radical having from 1 to 8 carbon atoms.

Some compounds of the formula I, such as, for example, tiliroside, can be isolated from plants, for example from plants of the genus Althaea, Aristolochia, Helianthemum, Lindera, Magnolia, Platanus, Potentlla, Quercus, Rosa, Sida, Sorbus and/or Tilia. These compounds can be processed further either in isolated form or in non-isolated form, i.e., for example, incorporated into cosmetic formulations in the form of an extract or in the form of a purified extract or alternatively in the form of the pure substance prepared from the plant extract. Of the said genera, the following species are preferred: Althaea officinalis, Althaea rosea, Aristolochia heterophylla, Helianthemum giomeratum, Lindera megaphylla, Magnolia salicifolia, Platanus acerifolia, Platanus occidentalis, Potentilla anserina, Quercus pubescens, Quercus suber, Quercus laurifolia, Quercus ilex, Quercus imbricaria, Quercus virginiana, Rosa pomifeta, Sida rhombifolia, Sida poeppigiana, Sida cordifolia, Sida glaziovii, Sorbus pendula, Tilia argenta and Tilia cordata.

If the cosmetic formulation according to the invention comprises tiliroside, this compound has, in a further preferred embodiment, been used in the form of a plant extract, a purified plant extract or in the form of the pure substance prepared from the plant extract for the preparation of the cosmetic formulation. In cosmetic formulations of this type, the plant extract comprises, for example, from 1 to 100% by weight of tiliroside. In one embodiment, the plant extract preferably comprises from 5 to 90% by weight of tiliroside. In a further embodiment, the plant extract preferably comprises from 30 to 100% by weight, particularly preferably from 60 to 100% by weight and especially preferably from 90 to 100% by weight of tiliroside. In a further preferred embodiment, the plant extract has been isolated by extraction of the Sida glaziovii plant

In all uses according to the invention in which tiliroside is used, for example if tiliroside is employed as UV filter, as free-radical scavenger and/or antioxidant, against oxidative stress, for preventing skin ageing or as active ingredient having an antiallergic, antiinflammatory, inflammation-inhibiting or antirritative action, in particular in cosmetic formulations, tiliroside can be used, for example, in the form of a synthetically prepared substance, in the form of a plant extract, a purified plant extract or an individual substance or in the form of a pure substance isolated from the plant extract. In a preferred embodiment, tiliroside is used in the form of a plant extract, a purified plant extract or in the form of the pure substance prepared from the plant extract.

The compounds of the formula I can be isolated or prepared by methods which are well known to the person skilled in the art and are described in the literature (for example in the standard works, such as Houben-Weyl, Methoden der organischen Chemie [Methods of Organic Chemistry], Georg-Thieme-Verlag, Stuttgart).

For example, tiliroside occurs in plants and can be isolated by extraction. The plant extracts are prepared by conventional methods of extraction of the plants or plant parts. Suitable extraction methods may be: maceration, remaceration, digestion, agitation rhaceration, fluidised-bed extraction, ultrasound extraction, countercurrent extraction, percolation, repercolation, evacolation, diacolation or solid/liquid extraction with continuous reflux, which is carried out in a Soxhlet extractor.

The solvent used for the extraction can be, for example, water or an alcohol.

It can be ascribed to the general knowledge of the person skilled in the art how these extractions can be carried out in detail and the resultant crude extracts can be purified by generally conventional methods.

One possible synthetic route for tiliroside is, for example, also described in B. Vermes, H. Wagner, Stud. Org. Chem. (Amsterdam) (1982), Volume date 1981, 11 (Flavonoids, Bioflavonoids), 161-167 and in B. Vermes, V. M. Chad, H. Wagner, Helv. Chim. Acta (1981), 64(4), 1964-1967.

The synthesis of tiliroside is shown in scheme 1.

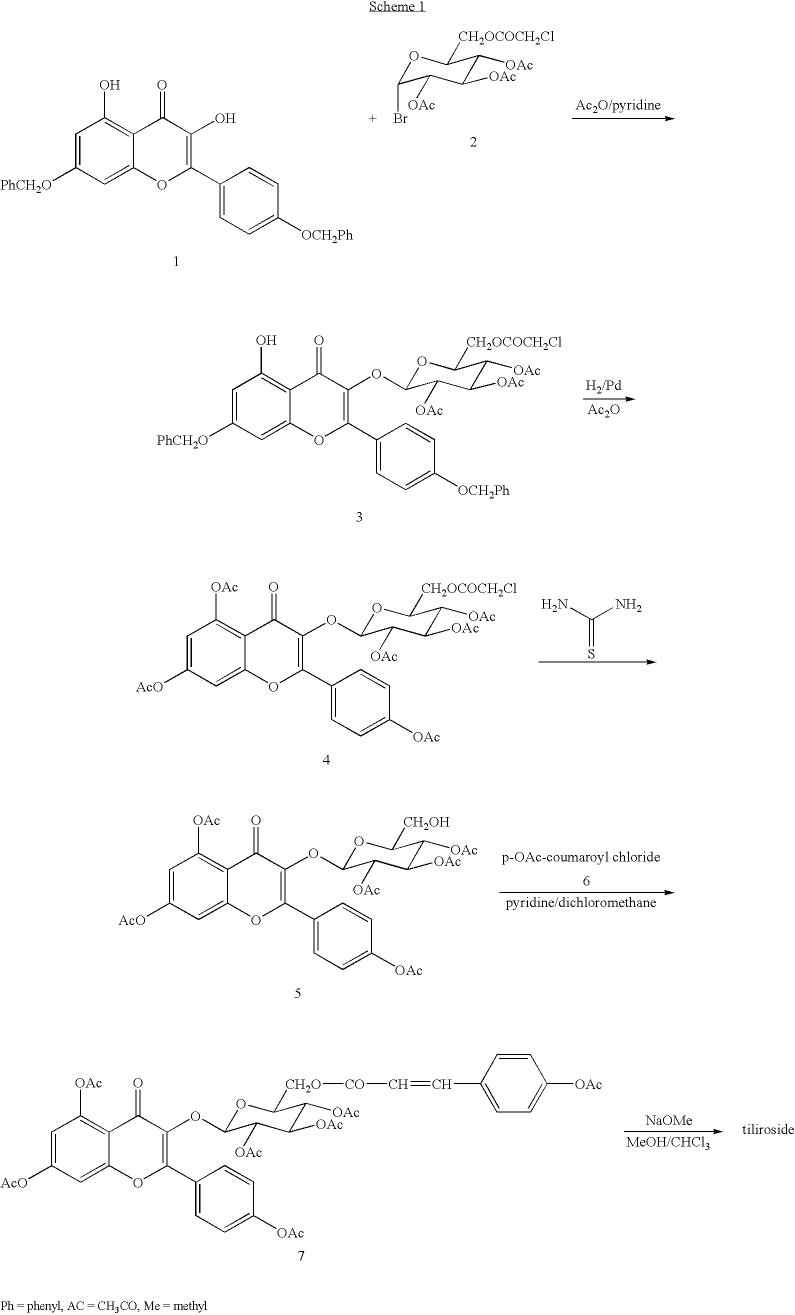

4′,7-Dibenzylkaempferol (1) [H. Wagner, H. Danninger, O. Seligmann, M. Nógrádi, L. Farkas, N. Farnsworth, Chem. Ber. 103 (1978) 3768] is reacted with 2,3,4-tri-O-acetyl-6-O-chloroacetyl-β-D-oucopyranosyl bromide (2) in the presence of Ag₂CO₃ and pyridine to give compound 3. Compound 2 can be prepared by the method described in D. Y. Gagniere, P. J. A. Wottero, Carbohydrate Res. 28 (1973) 1965. Catalytic debenzylation and subsequent careful acetylation of compound 3 gives compound 4, from which compound 5 can be obtained after removal of the chloroacetyl group using thiourea. In this compound, only one hydroxyl group is free, meaning that the esterificaton of compound 5 can proceed selectively. The esterification using the acid chloride p-acetylcoumaroyl chloride 6 can be carried out in a mixture of pyridine and dichloromethane. An excess of acid chloride and a long reaction time (about 96 hours) at room temperature are necessary to ensure that the esterification proceeds to completion The final step, the selective saponification of the 7 acetyl groups in compound 7, can be carried out by the method described in G. Zemplén, Chem. Ber. 59 (1926) 1258. This is carried out using a catalytic amount of NaOCH₃ and a calculated amount of methanol.

Other compounds of the formula I can be obtained by routine modification of the synthesis shown in scheme 1. Depending on the target molecule, different starting materials are used here, i.e. other optionally protected flavonoids, sugar components and radicals which are to be attached to the sugar component.

The esterification of glycosidic OH groups using aromatic sulfonic acid units can be carried out, for example, by the method described in A. B. Foster et al., J. Chem. Soc. (1954) 3625-3629. After this, the sugar component can, for example, be reacted with a corresponding aromatic sulfonyl chloride in pyridine.

The etherification of glycosidic OH groups using aromatic radicals can be carried out, for example, by the method described in P. Beraud et al.,

Tetrahedron Let. 30(3) (1989) 325-326. In this Mitsunobu reaction, the etherification is carried out, for example, by dissolving the sugar component in pyridine together with triphenylphosphine PPh₃ and reacting it with a corresponding phenol component and diethyl azodicarboxylate.

The etherification of glycosidic OH groups using radicals of saturated hydrocarbons can be carried out for example, by the method described in M. Goebel et al., Tetrahedron 53(9) (1997) 3123-3134. The etherification is carried out, for example, by carefully adding sodium hydride to the sugar component in dry dimethylformamide under an inert gas and then carefully reacting the mixture with a suitable alkylating reagent, such as, for example, a corresponding bromide.

The proportion of the compounds of the formula I in the cosmetic formulation is preferably from 0.001 to 20% by weight, particularly preferably from 0.01 to 10% by weight and especially preferably from 0.05 to 5% by weight, based on the cosmetic formulation as a whole. The proportion of the compounds of the formula I in the cosmetic formulation is very especially preferably from 0.05 to 2% by weight, based on the cosmetic formulation as a whole.

The protective action of the cosmetic formulations according to the invention against UV radiation can be improved if the formulation comprises one or more further UV filters in addition to the compounds of the formula I.

In principle, all UV filters are suitable for combination. Particular preference is given to UV filters whose physiological acceptability has already been demonstrated. Both for UV-A and UVB filters, there are many proven substances which are known from the specialist literature, for example

benzylidenecamphor derivatives, such as
3-(4′-methylbenzylidene)-dl-camphor (for example Eusolex® 6300),
3-benzylidenecamphor (for example Mexoryl® SD),
polymers of N-{(2 and 4)-[(2-oxobom-3-ylidene)methyl]benzyl}acrytamide (for example Mexoryl® SW),
N,N,N-trimethyl-4-(2-oxoborn-3-ylidenemethyl)aninium methylsulfate (for example Mexoryl® SK) or
α-(2-oxobom-3-ylidene)toluene-4-sulfonic acid (for example Mexoryl® SL),

benroyl- or dibenzoylmethanes, such as
1-(4-tert-butylphenyl)-3-(4-methoxyphenyl)propane-1,3-dione (for example Eusolex® 9020) or
4-isopropyldibenzoylmethane (for example Eusolex® 8020),

benzophenones, such as
2-hydroxy-4-methoxybenzophenone (for example Eusolex® 4360) or
2-hydroxy-4-methoxybenzophenone-5-sulfonic acid and its sodium salt (for example Uvinul® MS-40),

methoxycinnamic acid esters, such as
octyl methoxycinnamate (for example Eusolex® 2292),
isopentyl 4-methoxycinnamate, for example as a mixture of the isomers (for example Neo Heliopan® E 1000),

salicylate derivatives, such as
2-ethylhexyl salicylate (for example Eusolex® OS),
4-isopropylbenzyl salicylate (for example Megasol®) or
3,3,5-trimethylcyclohexyl salicylate (for example Eusolex® HMS),

4-aminobenzoic acid and derivatives, such as
4-aminobenzoic acid,
2-ethylhexyl 4-(dimethylamino)benzoate (for example Eusolex® 6007),
ethoxylated ethyl 4-aminobenzoate (for example Uvinul® P25),

benzimidazole derivatives, such as
2-phenylbenzimidazole-5-sulfonic acid and potassium, sodium and triethanolamine salts thereof (for example Eusolex® 232),
2,2′-(1,4-phenylene)bis(1H-benzimidazole-4,6-disulfonic acid, mono-sodium salt) (CAS No. 180 898-37-7),
2,2′-(1,4-phenylene)bis(1H-benzimidazole-5-sulfonic acid) and potassium, sodium and triethanolamine salts thereof,

and further substances, such as
2-ethylhexyl 2-cyano-3,3-diphenylacrylate (for example Eusolex® OCR),
3,3′-(1,4-phenylenedimethylene)bis(7,7dimethyl-2-oxobicyclo[2.2.1]-hept-1-ylmethanesuffonic acid and salts thereof (for example Mexoryl® SX),
2,4,6-trianilino-(p-carbo-2′-ethythexyl-1′-oxy)-1,3,5-triazine (for example Uvinul® T 150),
2-(2H-benzotriazol-2-yl)-4-methyl-6-(2-methyl-3-(1,3,3,3-tetramethyl-1-(trimethylsilyloxy)disiloxanyl)propyl)phenol (for example Silatrizole®),
2-ethylhexyl 4,4′-[(6-[4-((1,1-dimethylethyl)aminocarbonyl)phenyl-amino]-1,3,5-triazine-2,4-diyl)diimino]bis(benzoate) (for example Uvasorb® HEB),
α-(trimethylsilyl)-ω-[trimethylsilyl)oxy]poly[oxy(dimethyl [and about 6% of methyl[2-[p-[2,2-bis(ethoxycarbonyl]vinyl]phenoxyl-1-methyleneethyl] and approximately 1.5% of methyl[3-[p-[2,2-bis(ethoxycarbonyl)vinyl]-phenoxy)propenyl) and from 0.1 to 0.4% of (methylhydrogen]silylene]] (n≈60) (CAS No. 207 574-74-1),
2,2′-methylenebis(6-(2H-benzotriazol-2-yl)-4-(1,1,3,3-tetramethylbutyl)-phenol) (CAS No., 103 597-45-1),
2,4-bis{[4-(2-ethylhexyloxy)-2-hydroxy]phenyl}-6-(4-methoxyphenyl)-1,3,5-triazine (CAS No. 103 597-45-, -187 393-00-6).

The compounds listed should only be regarded as examples. It is of course also possible to use other UV filters. These organic UV filters, like the compounds of the formula I, are generally incorporated into cosmetic formulations in an amount of from 0.5 to 20% by weight, preferably in an amount of from 1 to 15% by weight and particularly preferably in amounts of from 2 to 8% by weight per individual substance. In total, the cosmetic preparations usually comprise up to 40% by weight, preferably from 5 to 25% by weight, of organic UV filters of this type.

Conceivable inorganic UV filters are those from the group consisting of titanium dioxides, such as, for example, coated titanium dioxide (for example Eusolex® T-2000, Eusolex® T-AQUA), zinc oxides (for example Sachtotec®), iron oxides and also cerium oxides. These inorganic UV filters are generally incorporated into cosmetic formulations in an amount of from 0.5 to 20% by weight, preferably from 2 to 10% by weight.

If different inorganic or organic UV filters are employed, these can be used in virtually any desired ratios to one another. The ratios of the individual substances to one another are usually in the range 1:10-10:1, preferably in the range 1:5-5:1 and particularly preferably in the range 1:2-2:1. If UV-A filters are employed alongside UV-B filters, it is advantageous for most applications for the proportion of UV-B filters to predominate and the ratio of UV-A filters:UV-B filters to be in the range from 1:1 to 1:10.

Besides the compounds of the formula I, preferred compounds having UV-filtering properties for cosmetic preparations are 3-(4′-methylbenzylidene)-di-camphor, 1-(4-tert-butylphenyl)-3-(4-methoxyphenyl)propane-1,3-dione, 4-isopropyidibenzoylmethane, 2-hydroxy-4-methoxybenzophenone, octyl, methoxycinnamate, 3,3,5-trimethylcyclohexyl salicylate, 2-ethylhexyl 4-(di-methylamirio)benzoate, 2-ethylhexyl 2-cyano-3,3-diphenylacrylate and coated titanium dioxide.

The protective action against oxidative stress or against the effect of free radicals can be further improved if the formulation comprises one or more further antioxidants.

There are many proven substances known from the specialist literature which can be used as antioxidants, for example amino acids (for example glycine, histidine, tyrosine, tryptophan) and derivatives thereof, imidazoles (for example urocanic acid) and derivatives thereof, peptides, such as D,L-camosine, D-camosine, L-camosine and derivatives thereof (for example anserine), carotinoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thio-dipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine) in very low tolerated doses (for example μmol to μmol/kg), and also (metal) chelating agents (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), vitamin A and derivatives (for example vitamin A palmitate), and coniferyl benzoate of benzoin resin, rutinic acid and derivatives thereof, α-glycosyl rutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Mixtures of antioxidants are likewise suitable for use in the cosmetic formulations according to the invention. Known and commercial mixtures are, for example, mixtures comprising, as active ingredients, lecithin, L-(+)-ascorbyl palmitate and citric acid (for example (for example Oxynex® AP), natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oynex® L LIQUID), DL-α-tocopherol, L-(+)-ascorbyl palmitate, citric acid and lecithin (for example Oxynex® LM) o r butylhydroxytoluene (BHT), L-(+)-ascorbyl palmitate and citric acid (for example Oxynex® 2004).

The proportion of the one or more antioxidants in the cosmetic formulation is preferably from 0.001 to 5% by weight, particularly preferably from 0.01 to 2% by weight, based on the formulation as a whole.

The protective action of the cosmetic formulations according to the invention against UV radiation and/or oxidative stress can also be improved if the formulation comprises one or more compounds selected from flavonoids and coumaranones in addition to the compounds of the formula I. Flavonoids are taken to mean the glycosides of flavonones, flavones, 3-hydroxyflavones (=flavonols), aurones, isoflavones and rotenoids [Römpp Chemie Lexikon [Römpp's Lexicon of Chemistry], Volume 9, 1993]. For the purposes of the present invention, however, this term is also taken to mean the aglycones, i.e. the sugar-free constituents, and the derivatives of the flavonoids and aglycones. For the purposes of the present invention, the term flavonoid is furthermore also taken to mean anthocyanidine (cyanidine). For the purposes of the present invention, the term coumaranones is also taken to mean the derivatives thereof.

Preferred flavonoids are derived from flavonones, flavones, 3-hydroxy-flavones, aurones and isoflavones, in particular from flavonones, flavones, 3-hydroxyflavones and aurones.

The flavonoids are preferably selected from the following compounds: 4,6,3′,4′-tetrahydroxyaurone, quercetin, rutin, isoquercetin, eriodictyol, taxifolin, luteolin, trishydroxyethylquercetin (troxequercetin), trishydroxyethyirutin (troxerutin), trishydroxyethylisoquercetin (troxeisoquercetin), trishydroxyethylluteolin (troxeluteolin) and the sulfates and phosphates thereof. Of the flavonoids, particular preference is given to rutin and troxerutin. Very especial preference is given to troxerutin.

Of the coumaranones, preference is given to 4,6,3′,4′-tetrahydroxybenzyl-3-coumaranone.

The proportion of the one or more compounds selected from flavonoids and coumaranones in the cosmetic formulation is preferably from 0.001 to 5% by weight, particularly preferably from 0.01 to 2% by weight, based on the formulation as a whole.

The formulations according to the invention may comprise vitamins as further ingredients. The cosmetic formulations according to the invention preferably comprise vitamins and vitamin derivatives selected from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopheroi E acetate, tocopherol hydrogensuccinate, vitamin K₁, esculin (vitamin P active ingredient), thiamine (vitamin B₁), nicotinic acid (niacin), pyridoxine, pyridoxal, pyridoxamine (vitamin B₆), pantothenic acid, biotin, folic acid and cobalamine (vitamin B₁₂), particularly preferably vitamin A palmitate, vitamin C, DL-α-tocopherol, tocopherol E acetate, nicotinic acid, pantothenic acid and biotin.

The formulations according to the invention may furthermore also comprise, as ingredient, ectoin [(S)-1,4,5,6-tetrahydro-2-methyl-4-pyrimidine-carboxylic acid] and then effect protection of skin cells, in particular protection of Langerhans cells. Cosmetic formulations comprising tiliroside and ectoin are particularly advantageous.

Addition of 1-(2-hydroxyaryl)alkan-1-one oximes (as described, for example, in EP 0 149 242) and preferably of 2-hydroxy-5-methyllaurophenone oxime provides the formulation according to the invention with an advantageous antiinflammatory action. Particularly advantageous are cosmetic formulations comprising tiliroside and 2-hydroxy-5-methyllaurophenone oxime in which the said substances are present in a weight ratio of from 1:10 to 10:1. Application forms of formulations of this type are, for example, aftersun preparations.

Preference is furthermore also given to formulations according to the invention which comprise tiliroside and 4,6,3′,4′-tetrahydroxybenzyl-3-coumaranone. The said substances are present in these formulations in a weight ratio of from 1:10 to 10:1.

Further active ingredients can also be incorporated into the formulations according to the invention, for example
hydroxyectoin [(S,S)-1,4,5,6-tetrahydro-5-hydroxy-2-methyl-4-pyrimidinecarboxylic acid]
active ingredients which can serve for wound treatment, such as, for example, allantoin
insect repellents, such as, for example, ethyl 3-[N-n-butyl-N-acetyl]-aminopropionate [CAS No. 52304-36-6]
sorbitol for skin care [for example Karion® F liquid or Karion® FP liquid]
biotin
anti-ageing products, such as, for example, mixtures comprising hydroxyproline or derivatives of hydroxyproline, for example mixtures comprising lecithin, hydroxyproline dipalmitate, sitosterol, linoleic acid, tocopherol, sodium ascorbate, mannitol, phenoxyethanol, methylparaben, ethylparaben, propylparaben, butylparaben, water [for example RonaCare™ ASC III® or, for example, mixtures comprising lecithin, hydroxylated lecithin, L-hydroxyproline, disodium rutinyl disulfate, phenoxyethanol, mannitol, magnesium ascorbyl phosphate, methylparaben, ethylparaben, propylparaben, butylparaben, sitosterol, tocopherol, sodium ascorbate, water [for example RonaCare™ VTA]
bisabolol.

The compounds of the formula I can be incorporated into cosmetic formulations in a conventional manner. Suitable formulations are those for external use, for example as a cream, lotion, gel or as a solution which can be sprayed onto the skin. It is preferred here for the preparation to comprise at least one oil phase and at least one water phase

Application forms of the cosmetic formulations according to the invention which may be mentioned are,for example: solutions, emulsions, PIT emulsions, suspensions, pastes, ointments, gels, creams, soaps, surfactant-containing cleansing preparations, lotions, oils, powders, sprays and aerosols. Further application forms are, for example, sticks, shampoos and shower products. In addition to the compounds of the formula I, any desired conventional excipients, adjuvants and optionally further active ingredients may be added to the formulation.

Preferred adjuvants originate from the group consisting of preservatives, antioxidants, stabilisers, solubilisers, vitamins, colorants, odour improvers, film formers, thickeners and humectants.

Solutions and emulsions can comprise the conventional excipients, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, groundnut oil, maize oil, olive oil, castor oil and sesame oil, glycerol fatty acid esters, polyethylene glycols and fatty acid esters of sorbitan, or mixtures of these substances.

The emulsions can exist in various forms. Thus, they can be, for example, an emulsion or microemulsion of the water-in-oil (W/O) type or of the oil-in-water (O/W) type, or a multiple emulsion, for example of the water-in-oil-in-water (W/O/W) type.

The cosmetic formulations may also be in the form of emulsifier-free, disperse preparations. They can be, for example, hydrodispersions or Pickering emulsions.

The cosmetic formulations may also be in the form of PIT emulsions or hydrogels. The cosmetic formulations may also comprise liposomes, which include, for example, active ingredients.

Suspensions can comprise the conventional excipients, such as liquid diluents, for example water, ethanol or propylene glycol, suspension media, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Pastes, ointments, gels and creams can comprise the conventional excipients, for example animal and vegetable fats, waxes, paraffins, starch, tragacanth, cellulose derivatives, polyethylene glycols, silicones, bentonites, silicic acid, talc and zinc oxide, or mixtures of these substances.

Soaps can comprise the conventional excipients, such as alkali metal salts of fatty acids, salts of fatty acid monoesters, fatty acid protein hydrolysates, isethionates, lanolin, fatty alcohol, vegetable oils, plant extracts, glycerol, sugars, or mixtures of these substances,

Surfactant-containing cleansing products can comprise the conventional excipients, such as salts of fatty alcohol sulfates, fatty alcohol ether sulfates, sulfosuccinic acid monoesters, fatty acid protein hydrolysates, isethionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils can comprise the conventional excipients, such as synthetic oils, such as fatty acid esters, fatty alcohols, silicone oils, natural oils, such as vegetable oils and oily plant extracts, paraffin oils, lanolin oils, or mixtures of these substances.

Powders and sprays can comprise the conventional excipients, for example milk sugar, talc, silicic acid, aluminium hydroxide; calcium silicate and polyamide powder, or mixtures of these substances. Sprays can additionally comprise the conventional propellants, for example chlorofluorocarbons, propane/butane or dimethyl ether.

Further typical cosmetic application forms are also lipsticks, lip-care sticks, mascara, eyeliner, eyeshadow, rouge, powder, emulsion and wax make-up as well as sunscreen, pre-sun and after-sun preparations.

All compounds or components which can be used in the cosmetic formulations are either known and commercially available or can be synthesised by known processes.

The cosmetic preparation according to the invention is particularly suitable for protecting the human skin against the harmful effects of the UV components in sunlight and also offer protection against ageing processes of the skin and against oxidative stress, i.e. against damage caused by free radicals, as are generated, for example, by exposure to sun, heat or other influences. It is in the form of various application forms usually used for this application. Thus, it can be, in particular, in the form of a lotion, or emulsion, such as a cream or milk (O/W, W/O, O/W/O, W/O/W), in the form of oily/alcoholic, oily/aqueous or aqueous/alcoholic gels or solutions, in the form of solid sticks or formulated as an aerosol.

The formulation may comprise cosmetic adjuvants which are usually used in preparations of this type, such as, for example, thickeners, plasticisers, humectants, surfactants, emulsifiers, preservatives, antifoaming agents, perfumes, waxes, lanolin, propellants, dyes and/or pigments which colour the agent itself or the skin, and other ingredients usually used in cosmetics.

The dispersant or solubiliser used can be an oil, wax or other fatty body, a lower monoalcohol or a lower polyol, or mixtures thereof. The particularly preferred monoalcohols or polyols include ethanol, i-propanol, propylene glycol, glycerol and sorbitol.

A preferred embodiment of the invention is an emulsion which is in the form of a protective cream or milk and, in addition to one or more compounds of the formula I and, if desired, further light-protection filters, comprises fatty alcohols, fatty acids, fatty acid esters, in particular triglycerides of fatty acids, lanolin, natural or synthetic oils or waxes and emulsifiers in the presence of water.

Further preferred embodiments are oily lotions based on natural or synthetic oils and waxes, lanolin, fatty acid esters, in particular triglycerdes of fatty acids, or oily/alcoholic lotions based on a lower alcohol, such as ethanol, or a glycol, such as propylene glycol, and/or a polyol, such as glycerol, and oils, waxes and fatty acid esters, such as triglycerides of fatty acids.

The cosmetic preparation according to the invention can also be in the form of an alcoholic gel comprising one or more lower alcohols or polyols, such as ethanol, propylene glycol or glycerol, and a thickener, such as siliceous earth. The oily-alcoholic gels additionally comprise natural or synthetic oil or wax.

Solid sticks consist of natural or synthetic waxes and oils, fatty alcohols, fatty acids, fatty acid esters, lanolin and other fatty bodies.

If a preparation is in the form of an aerosol, the conventional propellants, such as alkanes, fluoroalkanes and chlorofluoroalkanes, are generally used.

The cosmetic formulation may also be used to protect the hair against photochemical damage in order to prevent,colour changes, bleaching or damage of a mechanical nature. In this case, a suitable formulation is in the form of a rinse-out shampoo, lotion, gel or emulsion, the formulation in question being applied before or after shampooing, before or after colouring or bleaching or before or after permanent waving. It is also possible to select a formulation in the form of a lotion or gel for styling or treating the hair, in the form of a lotion or gel for brushing or blow-waving, in the form of a hair lacquer, permanent waving composition, colorant or bleach for the hair. Besides the compound(s) of the formula I and further UV filters, the cosmetic formulation may comprise various adjuvants used in this type of composition, such as surfactants, thickeners, polymers, softeners, preservatives, foam stabilisers, electrolytes, organic solvents, silicone derivatives, oils, waxes, antigrease agents, dyes and/or pigments which colour the composition itself or the hair, or other ingredients usually used for hair care.

The cosmetic preparations according to the invention can be prepared with the aid of techniques which are well known to the person skilled in the art.

For protection of the skin and/or natural or sensitised hair against sunlight, a cosmetic preparation comprising one or more compounds of the formula I is applied to the skin or hair. The term sensitised hair here is taken to mean hair which has been subjected to chemical treatment, such as permanent-wave treatment, a colouring process or a bleaching process.

The compounds of the formula I furthermore also have a stabilising action on the formulation. On use in the corresponding products, the latter therefore also remain stable for longer and do not change their appearance. In particular, the effectiveness of the ingredients, for example vitamins, is retained even on extended application or on extended storage. This is particularly advantageous in the case of compositions for protection of the skin against the action of UV rays since these cosmetics are subjected to particularly high stresses due to the UV radiation.

The invention furthermore relates to the stabilisation of UV filters. A known and effective class of light-protection filter substances is formed by the a dibenzoyimethane derivatives. However, it is disadvantageous that these substances are decomposed very easily by UV light and their protective properties are thus lost. An example of a commercially available light-protection filter from this class of compounds which may be mentioned is 4-(tert-butyl)4′-methoxydibenzoylmethane, which has the structure shown below.

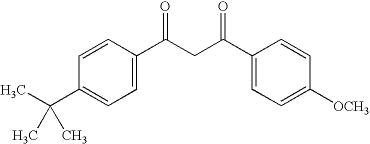

Surprisingly, it has now been found that compounds of the formula I have a very good stabilising action for the dibenzoylmethanes, in particular 4-(tert-butyl)-4-methoxydibenzoylmethane. A particularly high stabilising action has been found for tiliroside. Tiliroside can be used for stabilisation here as the pure substance or in the form of a plant extract. In a preferred embodiment, tiliroside is used in the form of a plant extract, a purified plant extract or in the form of the pure substance prepared from the plant extract. It is thus now possible to prepare light-protection agents using dibenzoylmethanes which exhibit only -a slight reduction in the protective action against UV rays, or none at all, even on extended exposure to the sun, for example during sunbathing for a number of hours.

The present invention furthermore relates to compounds of the formula I from claim 1 with the proviso that, in each case independently of one another, sulfate or phosphate is bonded to one or more hydroxyl groups of the radicals mentioned in the substituents Z₁ to Z₁₀ if Z₅ is a mono- or oligoglycoside radical to which one or more radicals selected from

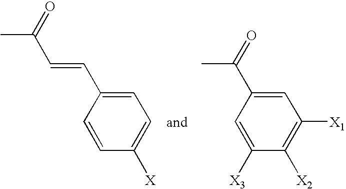

in which X, X₁, X₂ and X₃ are each, independently of one another, as defined in claim 1, are bonded, in each case via an —O-group. If one or more of the radicals X, X₁, X₂ and X₃ are OH, the said sulfate or phosphate groups may also be bonded to one or more of these hydroxyl groups. These compounds of the formula I are also referred to below as compounds of the formula I*.

In a preferred embodiment, Z₅ in the compounds of the formula I* is a mono- or oligoglycoside radical to which one or more radicals

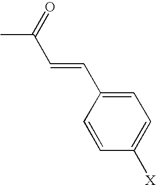

in which X is OH, CH₃COO, an alkoxy radical having from 1 to 8 carbon atoms or a monoglycoside radical, are bonded, in each case via an —O-group, and in which, in the said compounds, sulfate is bonded to one or more hydroxyl groups of the radicals mentioned in the substituents Z₁ to Z₁₀, in each case independently of one another. If X is OH, the said sulfate group may also be bonded to this hydroxyl group.

Of these compounds, particular preference is given to sulfated tiliroside, i.e. tiliroside which is characterised in that sulfate is bonded to one or more hydroxyl groups.

Compounds of the formula I, in particular of the formula I*, are also suitable as medicaments, for example in pharmaceutical formulations, where their above-mentioned advantageous actions, in particular as free-radical scavengers and/or antioxidants, are utilised. They act here, for example, in support of or in place of natural mechanisms which scavenge free radicals in the body. Compounds of the formula I, in particular of the formula I*, can in certain cases also be used, for example, for preventing certain types of cancer.

The invention furthermore relates to the use of the compounds of the formula I, in particular of the formula I*, and/or physiologically acceptable salts thereof for the preparation of pharmaceutical preparations, in particular by non-chemical methods. In this case, they can be brought into a suitable dosage form together with at least one solid, liquid and/or semi-liquid excipient or adjuvant and if desired in combination with one or more further active ingredients.

The invention furthermore relates to pharmaceutical preparations comprising at least one compound of the formula I, in particular of the formula I*, and/or one of its physiologically acceptable salts.

These preparations can be used as medicaments in human or veterinary medicine. Suitable excipients are organic or inorganic substances which are suitable for enteral (for example oral), parenteral or topical application and do not react with the compounds of the formula I, in particular of the formula I*, for example water, vegetable, oils, benzyl alcohols, alkylene glycols, polyethylene glycols, glycerol triacetate, gelatine, carbohydrates, such as lactose or starch, magnesium stearate, talc and Vaseline. Suitable for oral administration are, in particular, tablets, pills, coated tablets, capsules, powders, granules, syrups, juices or drops, suitable for rectal administration are suppositories, suitable for parenteral administration are solutions, preferably oil-based or aqueous solutions, furthermore suspensions, emulsions or implants, and suitable for topical application are ointments, creams or powders. The compounds of the formula I, in particular of the formula I*, may also be lyophilised and the resultant lyophilisates used, for example, for the preparation of injection preparations. The preparations indicated may be sterilised and/or comprise adjuvants, such as lubricants, preservatives, stabilisers and/or wetting agents, emulsifiers, salts for modifying the osmotic pressure, buffer substances, dyes, flavours and/or a plurality of further active ingredients, for example one or more vitamins.

The compounds of the formula I, in particular of the formula I*, are generally preferably administered in doses of between about 1 and 500 mg, in particular between 5 and 100 mg, per dosage unit. The daily dose is preferably between about 0.02 and 10 mg/kg of body weight. However, the specific dose for each patient depends on a very wide variety of factors, for example on the efficacy of the specific compound employed, on the age, body weight, general state of health, sex, on the diet, on the time and method of administration, on the excretion rate, medicament combination and severity of the particular disease to which the therapy applies.

The pharmaceutical formulations comprising one or more compounds of the formula I, in particular of the formula I*, can be prepared with the aid of techniques which are well known to the person skilled in the art.

The advantageous properties of the compounds of the formula I, in particular of the formula I*, can also be utilised, for example, when they are used in foods or as food supplements or as functional food. For example, the compounds of the formula I, in particular of the formula I*, can protect the other compounds present in the food, food supplement or functional food or even the organism against oxidation or against the action of free radicals.

The invention furthermore relates to foods which have been enriched with one or more compounds of the formula I, in particular of the formula I*, and to food supplements which comprise one or more compounds of the formula I, in particular of the formula I*.

The further explanations made regarding foods also apply analogously to food supplements and “functional food”. The foods which can be enriched with one or more compounds of the formula I, in particular of the formula I*, in accordance with the present invention include all materials which are suitable for consumption by animals or consumption by humans, for example vitamins and provitamins thereof, fats, minerals or amino acids. Foods which can be enriched with one or more compounds of the formula I, in particular of the formula I*, in accordance with the present invention are, for example, also foods which originate from a single natural source, such as, for example, sugar, unsweetened juice, squash or puree of a single plant species, such as, for example, unsweetened apple juice (for example also a mixture of different types of apple juice), grapefruit juice, orange juice, apple compote, apricot squash, tomato juice, tomato sauce, tomato puree, etc. Further examples of foods which can be enriched with one or more compounds of the formula I, in particular of the formula I*, in accordance with the present invention are corn or cereals from a single plant species and materials produced from plant species of this type, such as, for example, cereal syrup, rye flour, wheat flour or oatbran. Mixtures of foods of this type are also suitable for being enriched with one or more compounds of the formula I, in particular of the formula I*, in accordance with the present invention, for example multivitamin preparations, mineral mixtures or sweetened juice. As further examples of foods which can be enriched with one or more compounds of the formula I, in particular of the formula I*, in accordance with the present invention, mention may be made of food preparations, for example prepared cereals, biscuits, mixed drinks, foods prepared especially for children, such as yoghurt, diet foods, low-calorie foods or animal feeds.

The foods which can be enriched with one or more compounds of the formula I, in particular of the formula I*, in accordance with the present invention thus include all edible combinations of carbohydrates, lipids, proteins, inorganic elements, trace elements, vitamins, water and active metabolites of plants and animals.

The foods which can be enriched with one or more compounds of the formula I, in particular of the formula I*, in accordance with the present invention and the food supplements which comprise one or more compounds of the formula I, in particular of the formula I*, are preferably administered orally, for example-in the form of meals, pills, tablets, capsules, powders, syrup, solutions or suspensions.

The foods enriched with one or more compounds of the formula I, in particular of the formula I*, can be prepared with the aid of techniques which are well known to the person skilled in the art.

Even without further comments, it is assumed that a person skilled in the art will be able to utilise the above description in the broadest scope. The preferred embodiments should therefore merely be regarded as descriptive disclosure which is absolutely not to be regarded as limiting in any way.

The complete disclosure content of all applications and publications mentioned above and below is incorporated into this application by way of reference.

The following examples are intended to illustrate the present invention. However, they should in no way be regarded as limiting.

All compounds or components which can be used in the cosmetic formulations are either known and commercially available or can be synthesised by known methods.

The INCI names of the raw materials used are as follows,

| Raw material | INCI name |
|---|---|
| Abil WE 09 | Polyglyceryl 4-Isostearate, Cetyl |
| | Dimethicone Copolyol, Hexyl Laurate |
| Antaron V-220 | PVP/Eicosene Copolymer |
| Arlacel 80 | Sorbitan Oleate |
| Arlacel 165 V | Glyceryl Stearate, PEG-100 Stearate |
| Avocado oil | Persea Gratissima |
| Beeswax | Beeswax |
| Biobase ™ EP | Glyceryl Stearate, Cetearyl Alcohol, |
| | Sodium Stearoyl Lactylate, Lecithin |
| Carbopol ETD 2050 | Carbomer |
| Cetiol V | Decyl Oleate |
| Cetyl alcohol | Cetyl Alcohol |
| Cetyl isononanoate | Cetyl Isononanoate |
| Cutina HR | Hydrogenated Castor Oil |
| Dimeticon | Dimethicone |
| Eusolex ® 232 | Phenylbenzimidazole Sulfonic Acid |
| Eusolex ® 2292 | Octyl Methoxycinnamate, BHT |
| Eusolex ® 6300 | 4-Methylbenzylidene Camphor |
| Eusolex 8300 | 4-Methylbenzylidene |
| Eusolex ® 9020 | Butyl Methoxydibenzoylmethane |
| Eusolex ® HMS | Homosalate |
| Eusolex T-Aqua | Aqua (Water), Titanium Dioxide, |
| | Alumina, Sodium Metaphosphate, |
| | Phenoxyethanol, Sodium Methylparaben |
| Eutanol G | Octyldodecanol |
| Germaben II | Propylene Glycol, Diazolidinyl Urea, |
| | Methylparaben, Propylparaben |

| Raw material | INCI name |
|---|---|
| Germaben II-E | Propylene Glycol, Diazolidinyl Urea, |
| | Methylparaben, Propylparaben |
| Glycerin | Glycerin |
| Glycerin (87%) | Glycerin |
| Glycerin (87% extra pure) | Glycerin |
| Glycerin, anhydrous | Glycerin |
| Hetester PHA | Propylene Glycol Isoceteth-3 Acetate |
| Hexyl laurate | Hexyl Laurate |
| Imwitor 960 K flakes | Glyceryl Stearate SE |
| Isolan PDI | Diisostearoyl Polyglyceryl |
| | 3-Diisostearate |
| Isopropyl myristate | Isopropyl Myristate |
| Isopropyl palmitate | Isopropyl Palmitate |
| Jojoba oil | Buxus Chinensis (Jojoba Oil) |
| Karion F liquid | Sorbitol |
| Keltrol RD | Xanthan Gum |
| Magnesium sulfate | Magnesium Sulfate |
| Magnesium sulfate heptahydrate | Magnesium Sulfate |
| Methyl 4-hydroxybenzoate | Methylparaben |
| Miglyol 812 | Caprylic/Capric Triglyceride |
| Miglyol 812 N | Caprylic/Capric Triglyceride |
| Miglyol 812, neutral oil | Caprylic/Capric Triglyceride |
| Mirasil CM5 | Cyclomethicone |
| Mirasil DM 350 | Dimethicone |
| Montanov 68 | Cetearyl Alcohol, Cetearyl Glucoside |
| Sodium chloride | Sodium Chloride |
| Sodium hydroxide solution, 10% | Sodium Hydroxide |

| Raw material | INCI name |
|---|---|
| Oxynex ® K | PEG-8, Tocopherol, Ascorbyl |
| | Palmitate, Ascorbic Acid, Citric Acid |
| Panthenol-D | Panthenol |
| Paracera M | Microwax |
| Paraffin oil, liquid | Mineral Oil |
| Perfume oil TND-2417 | Perfume |
| Pemulen TR-1 | Acrylate/C₁₀₋₃₀ Alkyl Acrylate |
| | Crosspolymer |
| Pemulen ® TR-2 | Acrylate/C₁₀₋₃₀ Alkyl Acrylate |
| | Crosspolymer |
| Performa ® V 825 | Synthetic Wax |
| Polyglyceryl 2-dipolyhydroxy- | Polyglyceryl 2-Dipolyhydroxystearate |
| stearate | |
| Prisorine 2021 | Isopropyl Isostearate |
| 1,2-Propanediol | Propylene Glycol |
| Propyl 4-hydroxybenzoate | Propylparaben |
| Rhodicare S | Xanthan Gum |
| RonaCare ™ ASC III | Aqua, Lecithin, Dipalmitoyl |
| | Hydroxyproline, Phenoxyethanol, Tall |
| | Oil Sterol, Linoleic Acid, Tocopherol, |
| | Sodium Ascorbate, Mannitol, |
| | Methylparaben, Ethylparaben, |
| | Propylparaben, Butylparaben |
| RonaCare ™ Bisabolol | Bisabolol |
| RonaCare ™ Ectoin | Ectoin |
| RonaCare ™ LPO | Lauryl p-Cresol Ketoxime |
| RonaCare ™ Tocopherol acetate | Tocopheryl Acetate |
| Sepigel 305 | Polyacrylamide, C₁₃₋₁₄ Isoparaffin, |
| | Laureth-7 |

| Raw material | INCI name |
|---|---|
| SFE 839 | Cyclopentasiloxane, Dimethicone/ |
| | Vinyldimethicone Crosspolymer |
| Shea butter | Shea Butter |
| Steareth-2 | Steareth-2 |
| Steareth-10 | Steareth-10 |
| Stearic acid | Stearic Acid |
| DL-α-tocopherol acetate | Tocopherol Acetate |
| Triethanolamine | Triethanolamine |
| Triethanolamine extra pure | Triethanolamine |
| Water, demineralised | Aqua (Water) |
| Zinc stearate | Zinc Stearate |

### EXAMPLES

### Example A

Preparation of Sulfated Tiliroside, Sodium Salt

200 ml of water and 19.4 g of 32% sodium hydroxide solution (155.2 mmol) are added to 29.7 g of tiliroside (50 mmol) With stirring. 19.9 g of pyridine sulfone (125 mmol) are subsequently added, and the pH is adjusted to pH 8 by addition of 32% sodium hydroxide solution. The reaction batch is stirred under N₂ for 12 hours and then filtered, and the filtrate is concentrated to 50 g under reduced pressure (T=60° C.; p=100 mbar). 250 ml of methanol are added dropwise to the concentrated filtrate over the course of 1 hour, and the precipitated solid (sodium sulfate) is filtered off. Drying gives sulfated tiliroside, sodium salt.

### Example 1

Lotion (W/O) for application to the skin

| | | % by wt. |
|---|---|---|
| A | Polyglyceryl 2-Dipolyhydroxystearate | 5.0 |
| | Beeswax | 0.5 |
| | Zinc Stearate | 0.5 |
| | Hexyl Laurate | 9.0 |
| | Cetyl Isononanoate | 6.0 |
| | Shea Butter | 0.5 |
| | DL-α-Tocopherol Acetate | 1.0 |
| | Tiliroside | 0.5 |
| B | Glycerin | 5.0 |
| | Magnesium Sulfate Heptahydrate | 1.0 |
| | Preservatives | q.s. |
| | Water, Demineralised | to 100 |

Preparation

Phase A is warmed to 75° C. and phase B to 80° C. Phase B is added slowly to phase A with stirring. After homogenisation, the mixture is cooled with stirring. Perfumes are added at a temperature of 40° C.

The preservatives used are:

0.05% of propyl 4-hydroxybenzoate

0.15% of methyl 4-hydroxybenzoate

### Example 2

Lotion (W/O) for application to the skin

| | | % by wt. |
|---|---|---|
| A | Polyglyceryl 2-Dipolyhydroxystearate | 5.0 |
| | Beeswax | 0.5 |
| | Zinc Stearate | 0.5 |
| | Hexyl Laurate | 9.0 |
| | Cetyl Isononanoate | 6.0 |
| | Shea Butter | 0.5 |
| | DL-α-Tocopherol Acetate | 1.0 |
| B | Sulfated Tiliroside, Sodium Salt (Example A) | 1.0 |
| | Glycerin | 5.0 |
| | Magnesium Sulfate Heptahydrate | 1.0 |
| | Preservative | q.s. |
| | Water, Demineralised | to 100 |

Preparation

Phase A is heated to 75° C. and phase B to 80° C. Phase B is added slowly to phase A with stirring. After homogenisation, the mixture is cooled with stirring. Perfumes are added at a temperature of 40° C.

The preservatives used are the following:

0.05% of propyl 4-hydroxybenzoate

0.15% of methyl 4-hydroxybenzoate

### Example 3

Sunscreen spray (O/W)

| | | | % by wt. |
|---|---|---|---|
| A | Eusolex 9020 | (1) | 2.0 |
| | Eusolex ® HMS | (1) | 7.0 |
| | Steareth-2 | | 0.4 |
| | Steareth-10 | | 0.8 |
| | Pemulen ® TR-2 | (2) | 0.18 |
| | Hetester PHA | (3) | 5.0 |
| | Performa ® V 825 | (4) | 0.8 |
| | Dimeticon | | 1.0 |
| | Oxynex ® K | (1) | 0.1 |
| B | Sulfated Tiliroside, Sodium Salt (Example A) | | 1.0 |
| | Eusolex ® 232 | (1) | 1.0 |
| | Triethanolamine | (1) | 0.9 |
| | Propane-1,2-diol | (1) | 2.0 |
| | Water, Demineralised | | to 100 |

Preparation

Phase A

The constituents of phase A with the exception of Permulen® TR-2 are combined and warmed to 80° C. The Permuten® TR-2 is subsequently added with stirring.

Phase B

The water is mixed with the triethanolamine, and Eusolex® 232 is subsequently added with stirring. As soon as everything has dissolved, the other constituents of phase B are added, and the mixture is subsequently warmed to 80° C.

Preparation of the Sunscreen

Phase B is added slowly to phase A with stirring. After homogenisation, the mixture is cooled with stirring.

The preservatives used are:

0.05% of propyl 4-hydroxybenzoate

0.15% of methyl 4-hydroxybenzoate

Sources of Supply

(1) Merck KGaA

(2) BF Goodrich

(3) Bernel

(4) New Phase

### Example 4

Sunscreen cream (O/W)

| | | | % by wt. |
|---|---|---|---|
| A | Eusolex ® 2292 | (1) | 4.0 |
| | Eusolex ® 9020 | (1) | 1.0 |
| | Eusolex ® 6300 | (1) | 1.0 |
| | Stearic Acid | (1) | 2.5 |
| | Imwitor 960 K Flakes | (2) | 1.0 |
| | Antaron V-220 | (3) | 2.0 |
| | Cetyl Alcohol | (1) | 0.3 |
| | Miglyol 812 N | (4) | 5.5 |
| | Jojoba Oil | (5) | 2.0 |
| | Pemulen TR-1 | (6) | 0.25 |
| | Tiliroside | (1) | 1.0 |
| B | Glycerin, Anhydrous | (1) | 3.0 |
| | Propyl 4-Hydroxybenzoate | (1) | 0.1 |
| | Methyl 4-Hydroxybenzoate | (1) | 0.2 |
| | Keltrol RD | | 0.4 |
| | Water, Demineralised | | to 100 |
| C | Triethanolamine, Extra Pure | (1) | 0.9 |
| D | SFE 839 | (7) | 5.0 |
| | Arlacel 80 | (8) | 0.3 |

Preparation

Phase A is mixed and heated to 75° C. Phase B is mixed and heated to 70° C. Phase A is subsequently added to phase B, and the mixture is homogenised and cooled to 45° C. with stirring. Phase C and phase D are then added with stirring.

Sources of Supply:

(1) Merck KGaA

(2) Condea Chemie GmbH

(3) ISP Global Technologies

(4) Condea Chemie GmbH

(5) Gustav Heess GmbH

(6) BF Goodrich GmbH

(7) GE Silicones Holland

(8) Uniqema

### Example 5

Lotion (W/O) for application to the skin

| | | % by wt. |
|---|---|---|
| A | 4.6,3′,4′-Tetrahydroxybenzyl-3-coumaranone | 1.0 |
| | Polyglyceryl 2-Dipolyhydroxystearate | 5.0 |
| | Beeswax | 0.5 |
| | Zinc Stearate | 0.5 |
| | Hexyl Laurate | 9.0 |
| | Cetyl Isononanoate | 6.0 |
| | Shea Butter | 0.5 |
| | DL-α-Tocopherol Acetate | 1.0 |
| | Tiliroside | 1.0 |
| B | Glycerin | 5.0 |
| | Magnesium Sulfate Heptahydrate | 1.0 |
| | Preservatives | q.s. |
| | Water, Demineralised | to 100 |

Preparation

Phase A is warmed to 75° C. and phase B to 80° C. Phase B is added slowly to phase A with stirring. After homogenisation, the mixture is cooled with stirring. Perfumes are added at a temperature of 40° C.

The preservatives used are:

0.05% of propyl 4-hydroxybenzoate

0.15% of methyl 4-hydroxybenzoate

### Example 6

O/W after-sun lotion

| | | | % by wt. |
|---|---|---|---|
| A | RonaCare ™ Bisabolol | (1) | 0.3 |
| | Montanov 68 | (2) | 4.0 |
| | Miglyol 812, Neutral Oil | (3) | 12.0 |
| | Mirasil CM5 | (4) | 2.0 |
| | Mirasil DM 350 | (4) | 1.0 |
| | Tiliroside | | 1.0 |
| B | Water, Demineralised | | to 100 |
| | Glycerin (87% Extra Pure) | (1) | 3.0 |
| | Preservatives | | q.s. |
| | RonaCare ™ Ectoin | (1) | 1.0 |
| C | Rhodicare S | (4) | 0.5 |

Preparation

Phases A and B are heated separately to 75° C. Phase C is slowly added to phase B at 75° C. with stirring, and the mixture is stirred until a homogeneous mixture has formed. Phase A is subsequently added to the mixture B/C and homogenised. The resultant mixture is cooled to room temperature with stirring.

The preservatives used are:

0.05% of propyl 4-hydroxybenzoate

0.15% of methyl 4-hydroxybenzoate

Sources of Supply

(1) Merck KGaA

(2) Seppic

(3) Condea Chemie GmbH

(4) Rhodia GmbH

### Example 7

Sunscreen lotion (W/O)

| | | | % by wt. |
|---|---|---|---|
| A | Eusolex 8300 | (1) | 4.0 |
| | Eusolex 2292 | (1) | 7.0 |
| | Abil WE 09 | (2) | 5.0 |
| | Jojoba Oil | (3) | 3.0 |
| | Cetiol V | (4) | 3.0 |
| | Prisorine 2021 | (5) | 2.0 |
| | Paracera M | | 1.0 |
| | Miglyol 812, Neutral Oil | (6) | 3.0 |
| | Propyl 4-Hydroxybenzoate | (1) | 0.05 |
| | Tiliroside | | 1.0 |
| B | Eusolex T-Aqua | (1) | 16.0 |
| | Glycerin (87% Extra Pure) | (1) | 2.0 |
| | Sodium Chloride | (1) | 0.4 |
| | RonaCare ™ Ectoin | (1) | 1.0 |
| | Water, Demineralised | | to 100 |
| | Methyl 4-hydroxybenzoate | (1) | 0.15 |

Preparation

Phase B is heated to 80° C. and phase A to 75° C. Phase B is slowly stirred into phase A. The mixture is homogenised and cooled with stirring.

Sources of Supply

(1) Merck KGaA

(2) Goldschmidt AG

(3) Gustav Heess GmbH

(4) Cognis GmbH

(5) Uniqema

(6) Condea Chemie GmbH

### Example 8

A cream (O/W) comprising ectoin is prepared from the following components:

| | | | % by wt. |
|---|---|---|---|
| A | Paraffin, Liquid | (1) | 8.0 |
| | Isopropyl Myristate | (1) | 4.0 |
| | Mirasil CM5 | (2) | 3.0 |
| | Stearic Acid | (1) | 3.0 |
| | Arlacel 165 V | (3) | 5.0 |
| | Tiliroside | | 1.0 |
| B | Glycerin (87%) | (1) | 3.0 |
| | Germaben II | (4) | 0.5 |
| | Water, Demineralised | | to 100 |
| C | RonaCare ™ Ectoin | (1) | 1.0 |

Preparation

Firstly, phases A and B are warmed separately to 75° C. Phase A is then slowly added to phase B with stirring and stirring is continued until a homogeneous mixture has formed. After homogenisation of the emulsion, it is cooled to 30° C. with stirring. The mixture is subsequently warmed to 35° C., phase C is added, and the mixture is stirred until homogeneous.

Sources of Supply

(1) Merck KGaA

(2) Rhodia

(3) Uniqema

(4) ISP

### Example 9

Topical composition as W/O emulsion

| | | | % by wt. |
|---|---|---|---|
| A | Isolan PDI | (2) | 3.0 |
| | Paraffin Oil, Liquid | (1) | 17.0 |
| | Isopropyl Myristate | | 5.0 |
| | Beeswax | | 0.2 |
| | Cutina HR | (2) | 0.3 |
| | Tiliroside | | 1.0 |
| B | Water, Demineralised | | to 100 |
| | Glycerin (87%) | | 4.0 |
| | Magnesium Sulfate | | 1.0 |
| | Germaben II-E | (3) | 1.0 |
| C | RonaCare ™ LPO | (1) | 2.0 |

Preparation

Phases A and B are warmed to 75° C. Phase B is added to phase A with stirring. The mixture is subsequently homogenised using a Turrax for 2 minutes at 9000 rpm. The resultant mixture is cooled to from 30 to 35° C., and C is stirred in.

Sources of Supply

(1) Merck KGaA

(2) Goldschmidt AG

(3) ISP

### Example 10

After-sun lotion (O/W)

| | | | % by wt. |
|---|---|---|---|
| A | Biobase ™ EP | (4) | 4.5 |
| | Isopropyl Palmitate | (1) | 3.0 |
| | Cetiol V | (1) | 2.5 |
| | Miglyol 812 | (2) | 9.0 |
| | Carbopol ETD 2050 | (3) | 0.3 |
| | RonaCare ™ LPO | (2) | 1.0 |
| | Tiliroside | | 1.0 |
| B | Water, Demineralised | | to 100 |
| | Glycerin (87% Extra Pure) | (2) | 3.0 |
| | Preservatives | | q.s. |
| C | Sodium Hydroxide Solution, 10% | (2) | |

Preparation

Phases A and B are warmed separately to 70° C. Phase B is subsequently added to phase A with stirring. The mixture is then homogenised, neutralised using sodium hydroxide solution and cooled with stirring.

The preservatives used are:

0.05% of propyl 4-hydroxybenzoate

0.15% of methyl 4-hydroxybenzoate

Sources of Supply

(1) Cognis GmbH

(2) Merck KGaA

(3) BF Goodrich GmbH

(4) Tri-K Industries, Inc.

### Example 11

Skin-care gel (O/W)

| | | | % by wt. |
|---|---|---|---|
| A | Eusolex ® 6300 | (1) | 1.0 |
| | RonaCare ™ Tocopherol Acetate | (1) | 1.0 |
| | Avocado Oil | (2) | 5.0 |
| | Jojoba Oil | (2) | 5.0 |
| | Miglyol 812 N | (3) | 3.0 |
| | Eutanol G | (4) | 5.0 |
| | Sepigel 305 | (5) | 3.0 |
| | Tiliroside | (1) | 0.5 |
| B | Water, Demineralised | | to 100 |
| | Karion F Liquid | (1) | 5.0 |
| | Panthenol-D | (6) | 1.0 |
| C | RonaCare ™ ASC III | (1) | 4.0 |
| | Perfume Oil TND-2417 | (7) | 0.1 |
| | Preservatives | | q.s. |

Preparation

Phases A and B are pre-dissolved separately Phase B is subsequently added to phase A with stirring, and the constituents of phase C are added little by little.

The preservatives used are:

0.06% of propyl 4-hydroxybenzoate

0.15% of methyl 4-hydroxybenzoate

Sources of Supply

(1) Merck KGaA

(2) Gustav Heess GmbH

(3) Condea Chemie GmbH

(4) Cognis GmbH

(5) Interogana GmbH

(6) Hoffmann-La Roche AG

(7) Takasago

We claim:

1. A cosmetic formulation comprising at least one compound of formula I 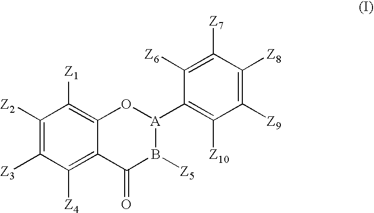
wherein
Z₁ to Z₄ and
Z₆ to Z₁₀ are each, independently of one another, H, OH, CH₃COO, alkoxy, hydroxyalkoxy, mono- or oligoglycoside radicals and wherein the alkoxy and hydroxyalkoxy groups are optionally branched or unbranched and comprise from 1 to 18 carbon atoms, 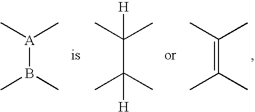
Z₅ is a mono- or oligoglycoside radical which comprises at least one radical which is 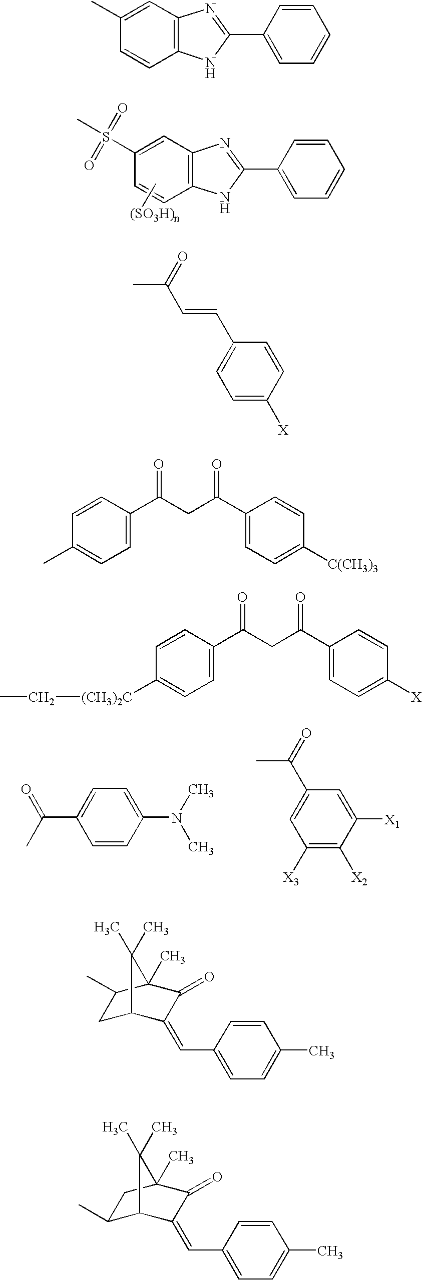 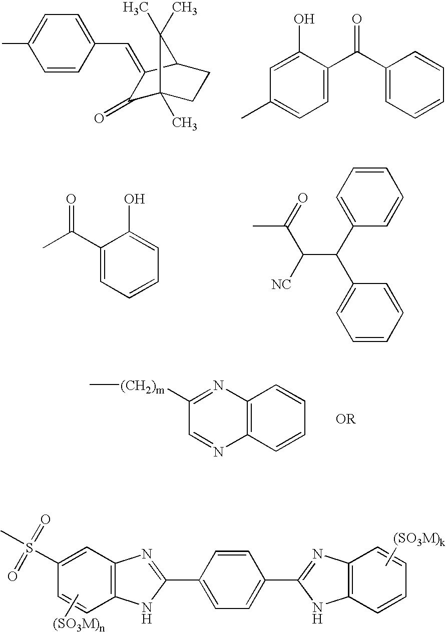
wherein
X, X₁, X₂ and X₃ are each, independently of one another, OH, CH₃COO, an alkoxy radical having from 1 to 8 carbon atoms or a monoglycoside radical,
n is 0, 1, 2 or 3, m is 0 or 1, k is 0, 1, 2, 3 or 4, and
M is H, Na or K,
is bonded to this glycoside radical, via an —O-group, and
wherein one or more hydrogen atoms in the OH group of the glycoside radicals in Z₁ to Z₁₀ are each, independently of one another, optionally replaced by one or more acetyl or by alkyl radicals having from 1 to 8 carbon atoms, and
wherein one or more hydroxyl groups in Z₁ to Z₁₀ optionally and independently of one another comprises a sulphate or a phosphate.

2. The cosmetic formulation according to claim 1, which comprises a compound of formula IA 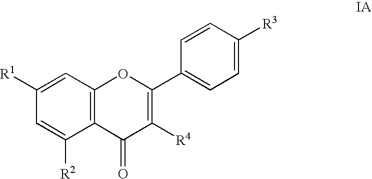
wherein
R¹, R² and R³ are each, independently of one another, OH, CH₃COO, an alkoxy radical having from 1 to 8 carbon atoms or a monoglycoside radical,
R⁴ is a mono- or diglycoside radical which comprises at least one group which is 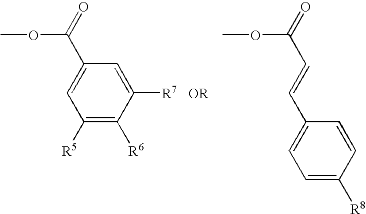
which is bonded to the glycoside radical via an —O-group,
R⁵, R⁶, R⁷
and R⁸ each, independently of one another, have the meaning of the radicals R¹ to R³, in each case bonded via an —O-group, and
in which one or more hydrogen atoms in the OH groups of the glycoside radical(s) may each, independently of one another, also be replaced by acetyl or by alkyl radicals having from 1 to 8 carbon atoms, and where, in each case independently of one another, sulfate or phosphate may also be bonded to one or more hydroxyl groups of the compounds of the formula IA.

3. The cosmetic formulation according to claim 2, which comprises a compound of formula IA1 or IA2

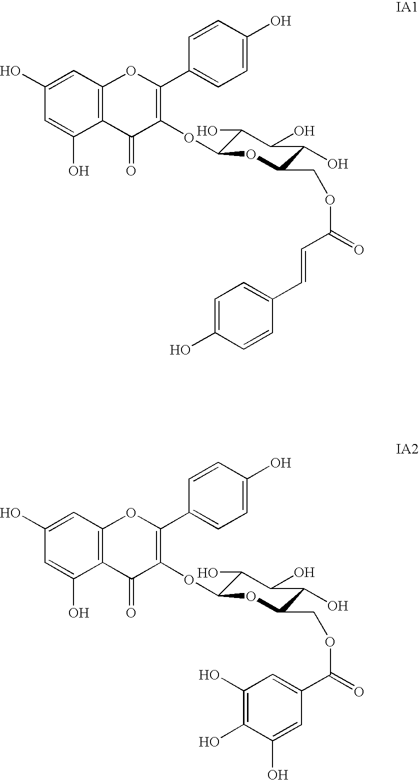

4. The cosmetic formulation according to claim 3, wherein the compound is of the formula IA1.

5. The cosmetic formulation according to claim 4, wherein the compound of the formula IA1 has been used in the form of a plant extract, a purified plant extract or in the form of the pure substance prepared from the plant extract for the preparation of the cosmetic formulation.

6. The cosmetic formulation according to claim 5, which is in the form of an emulsion.

7. The cosmetic formulation according to claim 5, wherein the compound is in the form of a plant extract isolated from the Sida glaziovii plant.

8. The cosmetic formulation according to claim 16, wherein the compound of the formula IA is present in the cosmetic formulation in an amount of from 0.001 to 20% by weight.

9. The cosmetic formulation according to claim 1, wherein the formulation comprises further UV filters.

10. The cosmetic formulation according to claim 9, wherein the further UV filter is selected from dibenzoylmethane and derivatives of dibenzoylmethane.

11. The cosmetic formulation according to claim 1, which comprises further antioxidants.

12. The cosmetic formulation according to claim 1, which comprises at least one additional compound which is a flavonoid or a coumaranone.

13. The cosmetic formulation according to claim 12, wherein the formulation comprises 4,6,3′,4′-tetrahydroxybenzyl-3 -coumaranone.

14. The cosmetic formulation according to claim 1, which comprises ectoin.

15. The cosmetic formulation according to claim 1, which further comprises 2-hydroxy-5-methyllaurophenone oxime.

16. A method of topically treating inflammation or filtering UV radiation comprising topically administering to a person in need thereof a cosmetic formulation comprising a compound of the formula I 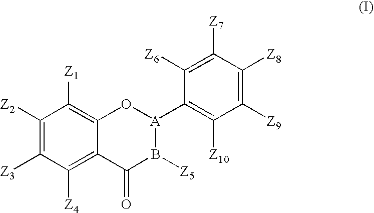
wherein
Z₁ to Z₄ and
Z₆ to Z₁₀ are each, independently of one another, H, OH, CH₃COO, alkoxy, hydroxyalkoxy, mono- or oligoglycoside radicals and wherein the alkoxy and hydroxyalkoxy groups are optionally branched or unbranched and comprise from 1 to 18 carbon atoms, 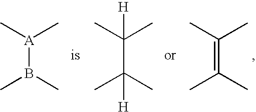
Z₅ is a mono- or oligoglycoside radical which comprises at least one radical which is 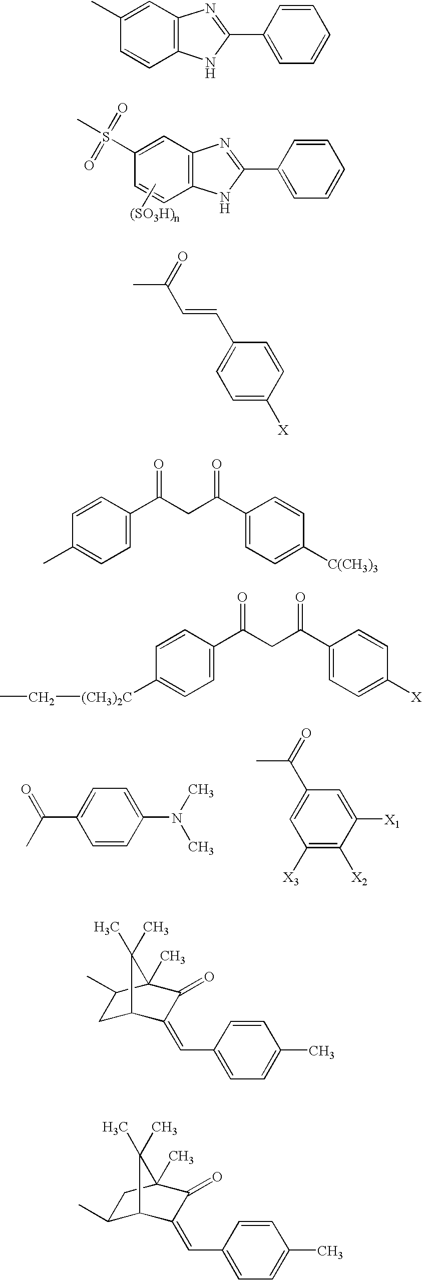 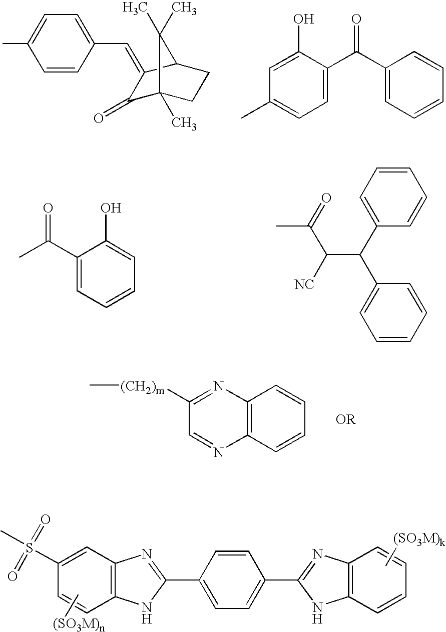
wherein
X, X₁, X₂ and X₃ are each, independently of one another, OH, CH₃COOO, an alkoxy radical having from 1 to 8 carbon atoms or a monoglycoside radical,
n is 0 or 1, 2, 3, m is or 1, k is 0, 1, 2, 3 or 4, and
M is H, Na or K,
is bonded to this glycoside radical via an —O-group, and
wherein one or more hydrogen atoms in the OH group of the glycoside radicals in Z₁ to Z₁₀ are each, independently of one another, optionally replaced by one or more acetyl or alkyl radicals having from 1 to 8 carbon atoms, and
wherein one or more hydroxyl groups in Z₁ to Z₁₀ optionally and independently of one another comprises a sulphate or a phosphate.

17. The method of claim 16 wherein said compound of the formula I also acts as a free-radical scavenger and/or antioxidant.

18. The method of claim 16 wherein said compound of the formula I also acts against oxidative stress.

19. The method of claim 16 wherein said compound of the formula I also delays skin ageing.

20. The method of claim 16 wherein said compound of the formula I also is antiallergic, inflammation-inhibiting or antirritative.

21. The method of claim 16 wherein said compound of the formula I also stabilizes UV filters.

22. The method according to claim 16, wherein the compound of the formula I is tiliroside.

23. The method according to claim 22, wherein tiliroside is in the form of a plant extract, a purified plant extract or in the form of the pure substance prepared from the plant extract.

24. The compound of formula I according to claim 1 with the proviso that, in each case independently of one another, sulfate or phosphate is bonded to one or more hydroxyl groups of the radicals mentioned in the substituents Z₁ to Z₁₀ if Z₅ is a mono- or oligoglycoside radical to which one or more radicals selected from 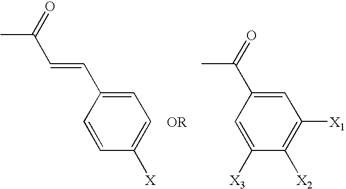
in which X, X₁, X₂ and X₃ are each, independently of one another, as defined in claim 1, are bonded, in each case via an —O-group.

25. A tiliroside compound wherein a sulfate is bonded to one or more hydroxyl groups.

26. A pharmaceutical composition comprising a carrier and at least one compound according to claim 24 or a physiologically accept salt thereof.

27. A food preparation which is enriched with at least one compound according to claim 24 or a physiologically acceptable salt thereof.

28. A food supplement which is enriched with at least one compound according to claim 24 or a physiologically acceptable salt thereof.

29. A method of topically treating inflammation or filtering UV radiation comprising topically administering to a person in need thereof a cosmetic formulation comprising a compound of the formula IA 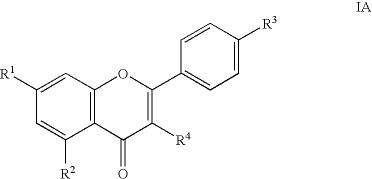
in which
R¹, R² and R³ are each, independently of one another, OH, CH₃COO, an alkoxy radical having from 1 to 8 carbon atoms or a monoglycoside radical,
R⁴ is a mono- or diglycoside radical, where 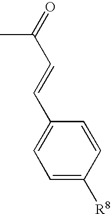
is bonded to the glycoside radical via an —O-group,
and R⁸ is OH, CH₃COO, an alkoxy radical having from 1 to 8 carbon atoms or a monoglycoside radical, and
in which one or more hydrogen atoms in the OH groups of the glycoside radical(s) may each, independently of one another, also be replaced by acetyl or by alkyl radicals having from 1 to 8 carbon atoms, and where, in each case independently of one another, sulfate or phosphate may also be bonded to one or more hydroxyl groups of the compounds of the formula IA,
wherein the compound of the formula IA is prepared synthetically or is in the form of a plant extract, a purified plant extract or in the form of the pure substance prepared from the plant extract,
and a cosmetically acceptable carrier for topical application to treat inflammation topically.
